(19) United States
(12) Patent Application Publication
(10) Pub. No.: US 2002/0025304 A1
(43) Pub. Date: Feb. 28, 2002

(54) Novel interferon for the treatment of multiple sclerosis

(76) Inventors: Edward M. Croze, Lafayette, CA (US); Daryl Faulds, Mill Valley, CA (US); T. Charis Wagner, Oakland, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/881,050

(22) Filed: Nov. 13, 2001

Related U.S. Application Data
(60) Provisional application No. 60/212,046, filed on Jun. 16, 2000.

(51) Int. Cl. A61K 38/21

(52) U.S. Cl. 424/85.6

(57) ABSTRACT

This invention relates to novel nucleic acids and polypeptide sequences, which code for an interferon-beta-2 (“IFN-β2”). A pharmaceutical composition, which comprises a pharmaceutically acceptable excipient and a therapeutically effective amount of a human IFN-β2 polypeptide, biologically-active fragment thereof, or biologically-active derivative thereof, is useful in treating multiple sclerosis in humans.

This application claims the benefit of the filing date of U.S. Provisional Application Serial No. 60/212,046, filed Jun. 16, 2000.

### BACKGROUND OF THE INVENTION

Interferons are intercellular signaling proteins that play an important role in variety of biological processes involving, e.g., cell proliferation and the immune response.

### BRIEF DESCRIPTION OF THE DRAWINGS

FIG. 1 shows the nucleotide sequence of human interferon-beta-2, including 5′ and 3′ sequences.

FIG. 2 shows the amino acid sequence of human interferon-beta-2. The translation of the open reading frame for IFN-β2 is shown. The signal sequence is shown italicized and the two potential N-glycosylation sites as well as the cysteines capable of forming a disulfide bond are shown underlined and in bold font.

FIG. 3 shows the three-dimensional structures of type I interferons. All three IFNs share a common 5-helical bundle motif characteristic of human type I IFNs. In addition, IFN-β1b and IFN-β2 are similar to each other in the location of their potential N-glycosylation sites and proposed disulfide bonds. A unique C-terminal amino acid sequence is present only in IFN-β2.

FIG. 4 is a protein alignment comparing human interferon-beta-2 to other interferon types.

FIG. 5 is a phylogenetic comparison of IFN-β2 to human type I and type II interferons. Based upon the pattern of cysteine conservation, potential N-glycosylation sites and a phylogenetic analysis, IFN-β2 is more closely related to IFN-β than to any of the other interferons.

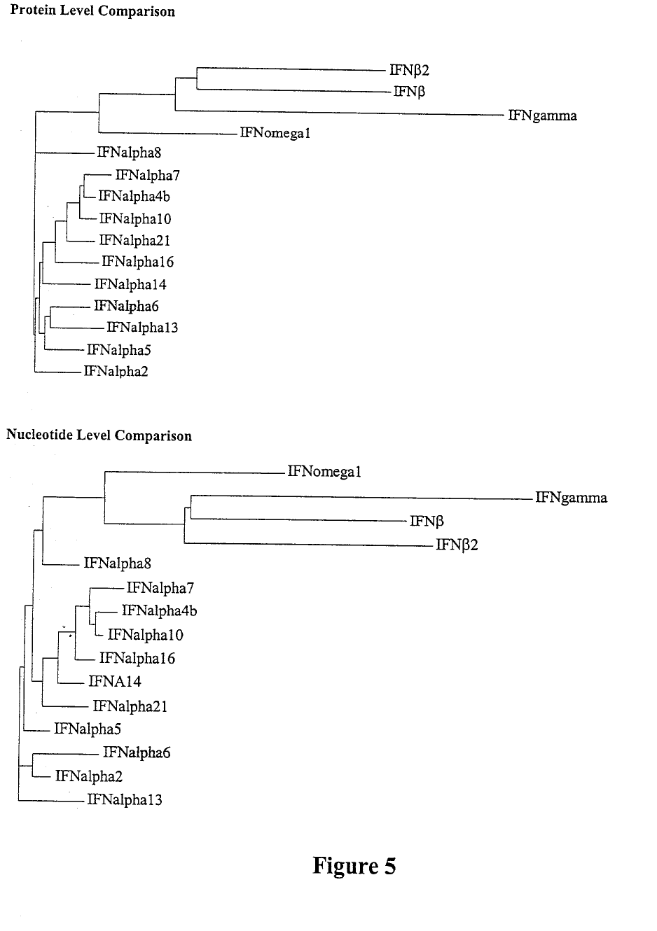

FIG. 6 shows a type I IFN dependent ISRE-luciferase reporter assay for a human interferon-beta-2.

FIG. 7 shows the results of inhibition of binding of IFN-β2 to human type I interferon receptor with an anti-IFN-β2 polyclonal antibody.

FIG. 8 (A and B) shows the effect of interferons on cell proliferation.

FIG. 9 illustrates the antiproliferative activities of IFN-β2 on human cells.

FIG. 10 shows the antiviral activity of interferons on human cells.

FIG. 11 shows competition between IFN-α2 and IFN-β2 for binding to the type I interferon receptor.

FIG. 12 is a 5′ genomic nucleotide sequence of human IFN-β2.

FIG. 13 is a nucleotide sequence coding for a 5′ region of human IFN-β2.

FIG. 14 is a 5′ polypeptide sequence of human IFN-β2.

FIG. 15 shows the antiproliferation of human fetal astrocytes in response to IFN-β2.

(A) is fetal brain culture 1 with no stimulation and (B) is fetal brain culture 1 with EGF stimulation; (C) is fetal brain culture 2 with no stimulation and (D) is fetal brain culture 2 with EGF stimulation.

### DESCRIPTION OF THE INVENTION

Novel nucleic acids, polypeptide sequences, and nucleic acid regulators thereof, have been identified which code for an interferon-beta-2 (“IFN-β2”), a class of intercellular signaling polypeptides which exert a myriad of biological effects, including, e.g., antitumor action, antiviral action, and immunoregulatory action. See, e.g., Cirelli and Tyring, Clin. Immunother. 3, 27-87, 1995. An interferon polypeptide of the present invention, fragments thereof, and derivatives thereof, have one or more of the following biological activities, including, but not limited to, IFN-β2 bioactivity, and an IFN-β2-specific immunogenic activity.

An “IFN-β2 bioactivity” means, e.g., functional effects, such as, alterations in cell membranes, anti-oncogene regulation, antitumor activity, antiviral activity, cell growth inhibition or antigrowth activity, anti-proliferation, enhancement of cytotoxicity of lymphocytes, immunoregulatory activity, inducement or inhibition of differentiation of target cells, macrophage activation, down-regulation of oncogenes, etc.; immunological effects, such as, reducing antibody formation, increasing cell membrane components (major histocompatability complex, Fc receptor, β2-microglobulin), modulating cell-mediated immunity, increasing cytokine (e.g., interleukin) production, increasing cytotoxic T cell effects, increasing macrophage effects, and increasing natural killing; interferon receptor binding activity, such as binding to interferon type I receptor, particularly an IFNAR2c chain thereof, and cellular effects stimulated by such receptor binding; activation and/or association with intracellular signaling molecules, such as Jak1, Tyk2, Stat1, Stat2, IRS-1, IRS-2, CrkL, CrkII, Vav, etc., and other downstream effectors (e.g., MAPK). See, e.g., Platanias and Fish, Exp. Hematology, 27:1583-1592, 1999.

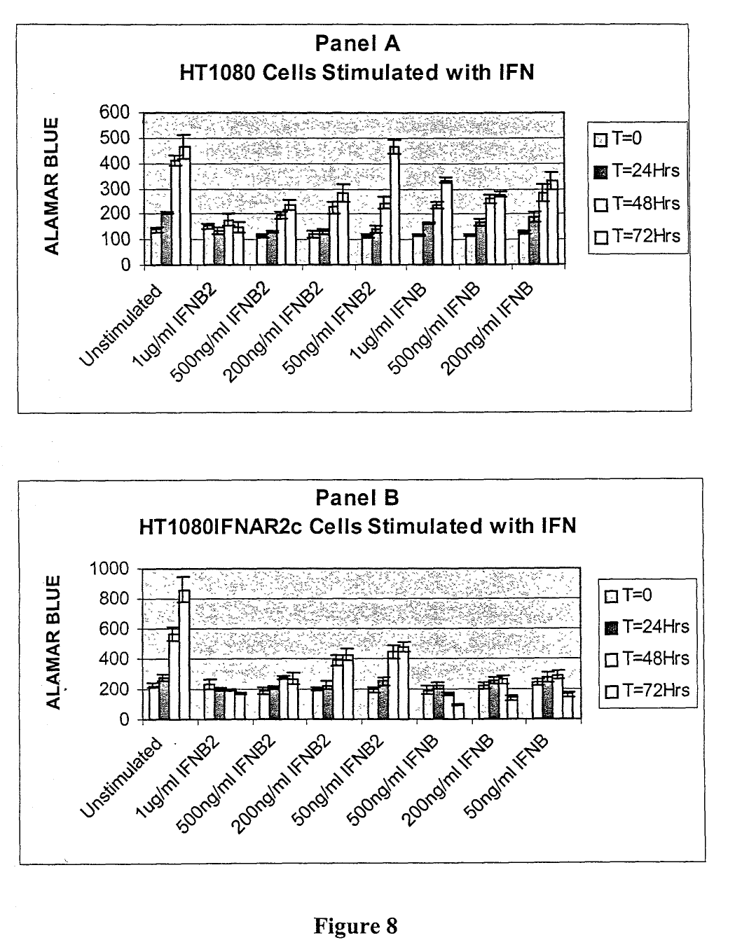

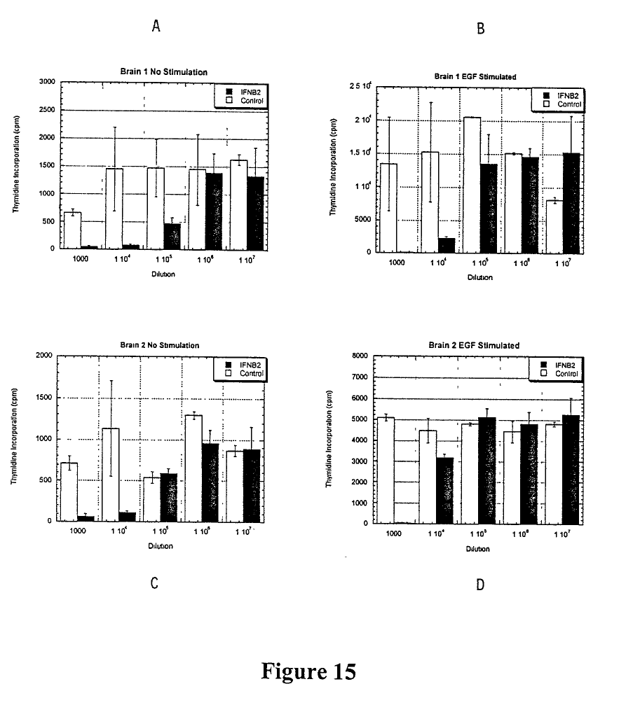

By the phrase “anti-proliferation activity”, it is meant that an IFN-β2 in accordance with the present invention inhibits cell growth, or induces apoptosis. This is illustrated in the Examples below. See, also, FIGS. 8, 9, and 15. For instance, IFN-β2 inhibits the proliferation of astrocytes in the brain. This activity is useful, e.g., in treating multiple sclerosis since astrocyte proliferation can lead to the neuronal inflammation which is characteristic of the disease. By inhibiting the growth of astrocytes, IFN-β2 reduces inflammation and ameliorates the disease. Thus, the present invention relates to methods of treating multiple sclerosis comprising administering amounts of IFN-β2 which are effective to inhibit astrocyte proliferation and/or reduce inflammation.

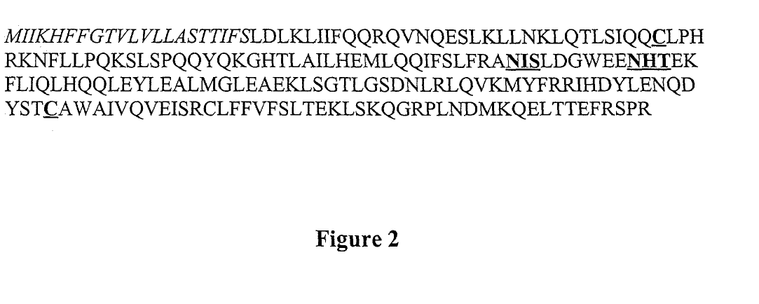

An “IFN-β2-specific immunogenic activity” means, e.g., that an IFN-β2 polypeptide elicits an immunological response which is selective or preferential for IFN-β2, e.g., an immunological response which is selective for mammalian IFN-β2. Thus, the stimulation of antibodies, T-cells, macrophages, B-cells, dendritic cells, etc., by an amino acid sequence selected from a mammalian IFN-β2, e.g., an IFN-β2 in FIG. 2, is a specific immunogenic activity. These responses can be measured routinely.

IFN-β2 is a full-length mammalian polypeptide having an amino acid sequence which is obtainable from a natural source and which has one or more of the aforementioned bioactivities. It can have a sequence as shown in FIG. 2, having an open-reading frame that begins with an initiation codon and ends with a stop codon. It includes naturally occurring normal, naturally occurring mutant, and naturally occurring polymorphic, including single nucleotide polymorphisms (SNP), etc., sequences. Natural sources include, e.g., living cells, e.g., obtained from tissues or whole organisms, cultured cell lines, including primary and immortalized cell lines, biopsied tissues, etc.

The present invention also relates to fragments of a mammalian IFN-β2. The fragments are preferably “biologically active”. By “biologically active”, it is meant that the polypeptide fragment possesses an activity in a living system or with components of a living system. Biological activities include those mentioned, e.g., IFN-β2-bioactivity and IFN-β2-specific immunogenic activity. Fragments can be prepared according to any desired method, including, chemical synthesis, genetic engineering, cleavage products, etc. A biologically active fragment of an IFN-β2 includes polypeptides which have had amino acid sequences removed or modified at either the carboxy- or amino-terminus of the protein.

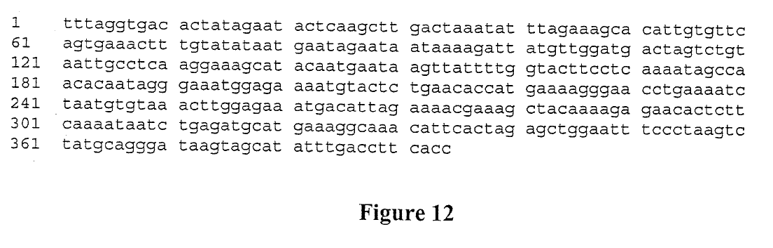

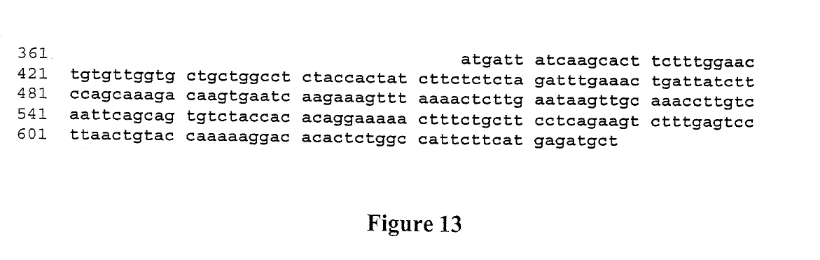

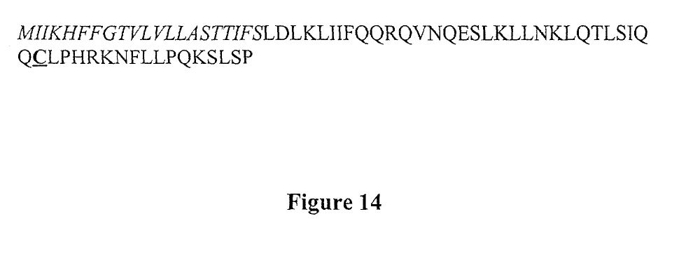

Preferably, the nucleic acids, and fragments thereof, in FIGS. 12 and 13 are excluded. Preferably, the polypeptides, and fragments thereof, in FIG. 14 are excluded. However, polypeptides which contain or comprise these sequences are not excluded, e.g., full-length IFN-β2, a polypeptide having two or more of these mentioned fragments, or a polypeptide having one of the mentioned fragments and additional amino acid sequences, either from IFN-β2 or from another source.

The present invention also relates to an IFN-β2 having a deduced sequence of amino acids 1 to 208 as shown in FIG. 2. Its calculated molecular weight is about 23,000 daltons and the predicted isoelectric point is 8.1. It is an acid stable protein having a molecular weight of about 26,000 daltons as measured by SDS-PAGE. The unglycosylated form produced in E. coli has a molecular weight of about 20,500 daltons. See, Examples below.

As shown in FIG. 2, it has a predicted signal sequence from amino acid -1 to -21, two potential N-glycosylation sites at amino acids 74-77 and 83-86, and cysteine residues at 32, 142, and 154. A disulfide bond is predicted to form between cysteine residues 32 and 142. It comprises a helix A at amino acid positions 7-24, helix B at 55-69, helix C at 83-95, helix D at 118-134, and helix E at 143-158.

Mature IFN-β2 refers to an IFN-β2 which lacks amino acids from -1 to -21 as shown in FIG. 2 and is 187 amino acids in length. It also has a unique 18 amino acid extension at the C-terminus when compared to other known interferon types. Such extension can be used as marker for IFN-β2, at both nucleotide and amino acid level, and can be fused to heterologous polypeptides.

An IFN-β2 polypeptide of the invention, e.g., having an amino acid sequence as shown in FIG. 2, can be analyzed by any suitable methods to identify other structural and/or functional domains in the polypeptide, including membrane spanning regions, hydrophobic regions. For example, an IFN-β2 polypeptide can be analyzed by methods disclosed in, e.g., Kyte and Doolittle, J. Mol. Biol., 157:105, 1982; EMBL Protein Predict; Rost and Sander, Proteins, 19:55-72, 1994.

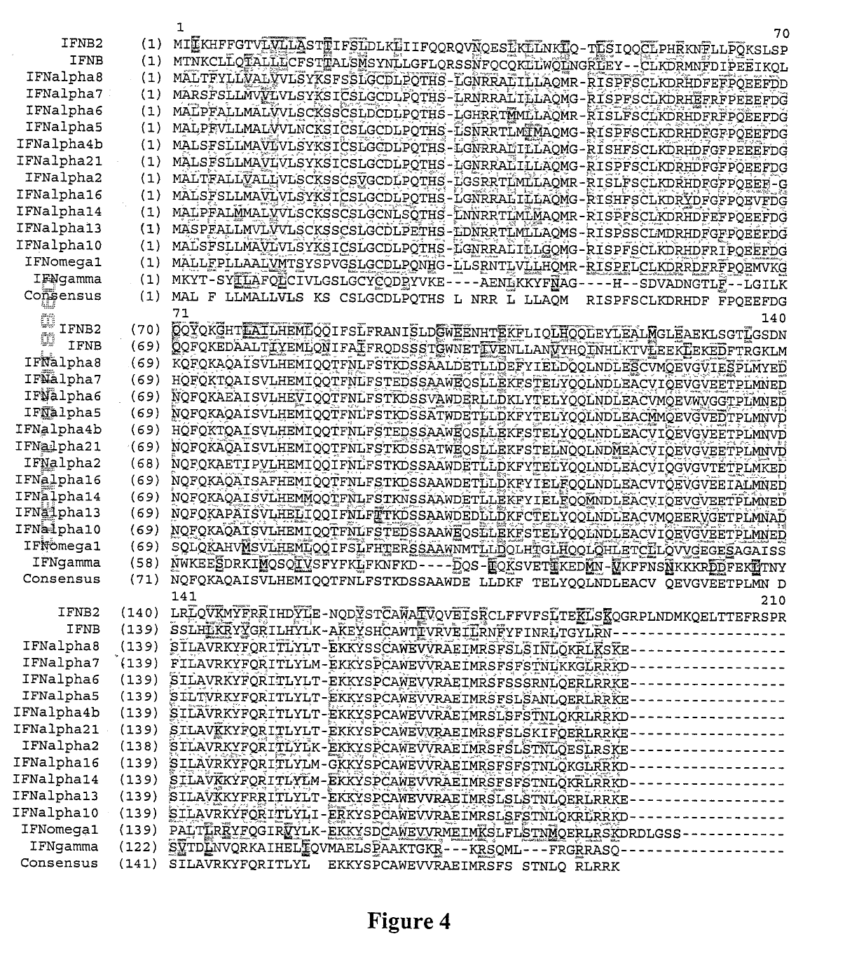

Other homologs of IFN-β2s of the present invention can be obtained from mammalian and non-mammalian sources according to various methods. For example, hybridization with oligonucleotides (e.g., primers to amplify the coding region -5′-ATG ATT ATC AAG CAC TTC TTT GGA-3′ and 5′-CTA CCT CGG GCT TCT AAA CTC TGT-3′). Primers used for expression in E. coli- 5′-GGA ATT CCT ACT ACC TCG GGC TTC TAA-3′ and 5′-GCG CGC GCA TAT GCT AGA TTT GAA ACT GAT TAT-3′. Primers for the full length known sequence including 5′ and 3′ untranslated genomic sequence-5′-TTT AGG TGA CAC TAT AGA AT-3′ and 5′-TAA AAT GGA TAG AAT ATA TAA-3′-can be employed to select homologs, e.g., as described in Sambrook et al., Molecular Cloning, Chapter 11, 1989. Such homologs can have varying amounts of nucleotide and amino acid sequence identity and similarity to IFN-β2. Mammalian organisms include, e.g., rodent, mouse, rat, hamster, monkey, ape, pig, cow, horse, dog, cat, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, Crustacea, artemia, viruses, etc. To select oligonucleotides for hybridization, an effective method can be used. For example, IFN-β2-specific regions can be identified by comparing an IFN-β2 of the present invention with other IFN-β2 types and selecting those amino acid sequences which only appear in the former (i.e., nonconserved, or, “specific-for” IFN-β2). See, e.g., FIG. 4 showing conserved and non-conserved regions between the different interferon types. Non-conserved amino acid sequences can be chosen (e.g., KSLSP) and degenerate probes can be designed based on such sequences. See, also, Venkataraman et al., Proc. Natl. Acad. Sci., 96:3658-3663, 1999. Other specific (i.e., non-conserved) and/or conserved amino acid sequences can be found routinely, e.g., by searching a gene/protein database using the BLAST set of computer programs.

The invention also relates to IFN-β2-specific amino acid sequences, e.g., a defined amino acid sequence which is found in the particular sequence of FIGS. 2 and 4, but not in other interferon types. Preferred polypeptides are at least about eight contiguous amino acids, e.g., about 9, 10, 12, 15, 20, 21, 22, 25, 30, 40, 50, etc. Such polypeptides can comprise, e.g., KHFFGTV, IIFQQRQV, KSLSP, FRANI, AEKLSGT, CLFFVFS, and QGRPLNDMKQELTTEFRSPR, and fragments thereof. An IFN-β2-specific amino acid sequence or motif can be useful to produce peptides as antigens to generate an immune response specific for it. Antibodies obtained by such immunization can be used as a specific probe for a mammalian IFN-β2 protein for diagnostic or research purposes, including as expression markers.

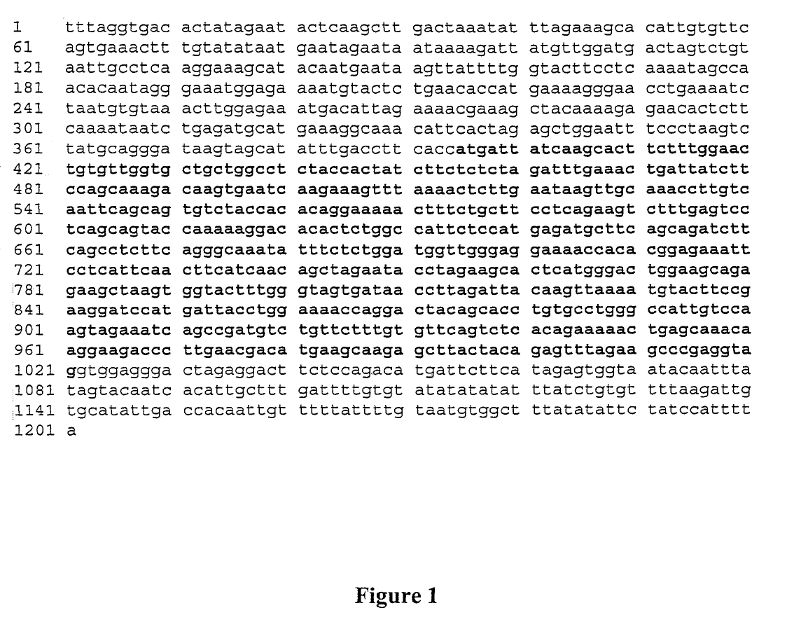

As mentioned, polypeptides of the present invention can comprise various amino acid sequences for an IFN-β2 (e.g., a full-length sequence, i.e., having a start and stop codon as shown in FIG. 1, a mature amino acid sequence (i.e., where the IFN-β2 polypeptide is produced as a precursor which is processed into a mature polypeptide, or fragments thereof). Useful fragments include, e.g., fragments comprising, or consisting essentially of, any of the aforementioned domains and specific or conserved amino acid sequences such as those shown in FIGS. 2 and 4.

A fragment of an IFN-β2 polypeptide of the present invention can be selected to have a specific biological activity, e.g., antiviral, immunomodulatory, antigrowth, etc. The measurement of these activities can be performed as known. See, below. These peptides can also be identified and prepared as described in EP 496 162.

A polypeptide of the present invention can also have 100% or less amino acid sequence identity to the amino acid sequence set forth in FIG. 2. For the purposes of the following discussion, sequence identity means that the same nucleotide or amino acid which is found in the sequence set forth in FIGS. 1 and 2 is found at the corresponding position of the compared sequence(s). A polypeptide having less than 100% sequence identity to the amino acid sequences set forth in FIGS. 1 and 2 can contain various substitutions from the naturally occurring sequence, including homologous and non-homologous amino acid substitutions. See below for examples of homologous amino acid substitution. The sum of the identical and homologous residues divided by the total number of residues in the sequence over which the IFN-β2 polypeptide is compared is equal to the percent sequence similarity. For purposes of calculating sequence identity and similarity, the compared sequences can be aligned and calculated according to any desired method, algorithm, computer program, etc., including, e.g., FASTA, BLASTA.

A polypeptide having less than 100% amino acid sequence identity to the amino acid sequence of FIG. 2 can have about 99%, 98%, 97%, 96%, 95%, 94%, 93%, 92%, 91%, 90%, 88%, 85%, 80%, 75% 70%, or as low as about 53% sequence identity.

The present invention also relates to polypeptide muteins of an IFN-β2, i.e., any polypeptide which has an amino acid sequence which differs in amino acid sequence from an amino acid sequence obtainable from a natural source (a fragment of a mammalian IFN-β2 does not differ in amino acid sequence from a naturally occurring IFN-β2 although it differs in amino acid number). Thus, IFN-B2 polypeptide muteins comprise amino acid substitutions, insertions, and deletions, including non-naturally occurring amino acids.

Muteins to an IFN-β2 amino acid sequence of the invention can also be prepared based on homology searching from gene data banks, e.g., Genbank, EMBL. Sequence homology searching can be accomplished using various methods, including algorithms described in the BLAST family of computer programs, the Smith-Waterman algorithm, etc. A mutein(s) can be introduced into a sequence by identifying and aligning amino acids within a domain which are identical and/or homologous between polypeptides and then modifying an amino acid based on such alignment. For instance, IFN-β2 of the present invention shares sequence identity with various known interferons as shown in FIG. 4. Alignments between these polypeptides at conserved amino acid residues can identify residues whose modification would be expected to reduce, decrease, or, eliminate a biological activity of an IFN-β2, such as a receptor binding activity, etc. For instance, where alignment reveals identical amino acids conserved between two or more domains, elimination or substitution of the amino acid(s) would be expected to adversely affect its biological activity.

Amino acid substitution can also be made by replacing one homologous amino acid for another. Homologous amino acids can be defined based on the size of the side chain and degree of polarization, including, small nonpolar: cysteine, proline, alanine, threonine; small polar: serine, glycine, aspartate, asparagine; large polar: glutamate, glutamine, lysine, arginine; intermediate polarity: tyrosine, histidine, tryptophan; large nonpolar: phenylalanine, methionine, leucine, isoleucine, valine. Homologous acids can also be grouped as follows: uncharged polar R groups, glycine, serine, threonine, cysteine, tyrosine, asparagine, glutamine; acidic amino acids (negatively charged), aspartic acid and glutamic acid; basic amino acids (positively charged), lysine, arginine, histidine. Homologous amino acids also include those described by Dayhoff in Atlas of Protein Sequence and Structure 5, 1978, and by Argos in EMBO J., 8, 779-785, 1989.

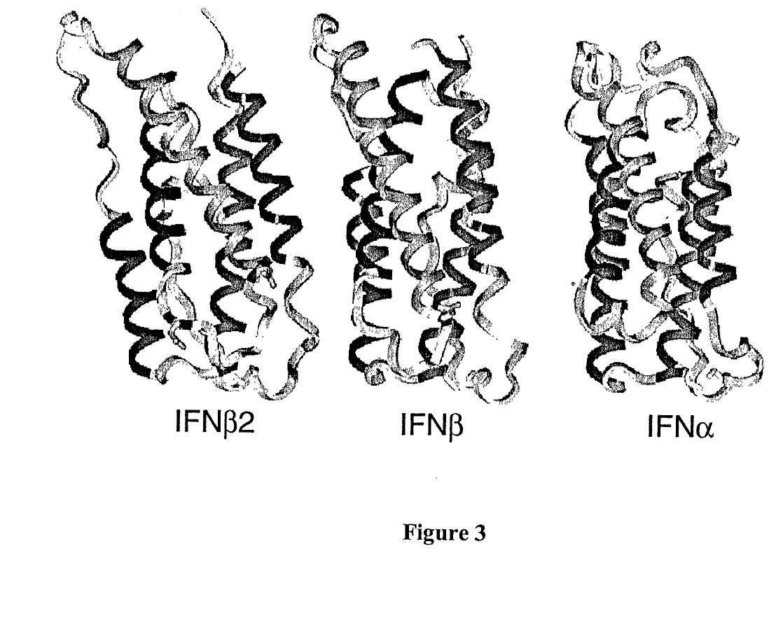

Muteins can be prepared which have no effect on activity, or which reduce or increase the activity of IFN-β2 as compared to the wild-type. Mutations can be made in analogy to other IFN-β2s, such as fibroblast interferon (beta-1) and alpha-interferons. For example, IFN-β2 folds in the characteristic five helical bundles typical for interferon: helix A at amino acid positions 7-24, helix B at 55-69, helix C at 83-95, helix D at 118-134, and helix E at 143-158. These helixes are tethered together by random coil or loop regions. See, FIG. 3. Mutations to the helical structures can be made in analogy to IFN-β1 (fibroblast), e.g., as described in Runkel et al., Biochemistry, 39:2538-2551, 2000. For instance, parts of the A helix, the AB loop, and the E helix are involved in receptor binding. See, also, Piehler and Schreiber, J. Mol. Biol., 294:223-237, 1999.

The IFN-β2 of the present invention, like fibroblast interferon, has three cysteine residues at amino acid positions 32, 142, and 154. The cysteine residues at positions 32 and 142 are similar in location to the cysteine residues known to form a disulfide bond in fibroblast interferon. In analogy to fibroblast interferon, the amino acid at position 154 can be deleted or substituted with a neutral amino acid, such as glycine, valine, alanine, leucine, isoleucine, tyrosine, phenylalanine, histidine, tryptophan, serine, threonine, or methionine. Serine and threonine are preferred because of their chemical similarity to cysteine. Removal of the unpaired cysteine can prevent the formation of incorrect intramolecular and intermolecular disulfide bonds. See, e.g., U.S. Pat. No. 4,588,585. Mutations can also be made in accordance with U.S. Pat. Nos. 4,914,033, 5,545,723, and 5,580,723. See, also, e.g., Fish et al., J. Interferon Res., 12:257-66, 1992; Fish et al., J. Interferon Res., 9:97-114, 1989; DiMarco et al., J. Interferon Res., 13:139, 1993; Mitsui et al., Pharmacol. Therap., 58:93-132, 1993; Wang et al., J. Immunol., 152:705-715, 1994.

The invention relates to mutein nucleic acids coding for such mutein polypeptides. Thus, the present invention relates to nucleotide sequences of FIG. 1, wherein said nucleic acids code for a polypeptide and one or more amino acid positions are substituted or deleted, or both, and the polypeptide coded for by the nucleic acid has a biological activity, such as enhanced recovery from bacteria cells when expressed recombinantly, or enhanced bioactivity. A polypeptide mutein, and its corresponding nucleotide coding sequence, can have an amino acid sequence as set forth in FIG. 2 except where one or more positions are substituted by homologous amino acids, e.g., where there are 1, 5, 10, 15, or 20 substitutions. How a modification affects the mentioned activities can be measured according to the methods described above, below, and as the skilled worker in the field would know.

Various methods of assaying for IFN-β2 activity are known. For example, useful assays include: antiviral assays, such as CPE (e.g., U.S. Pat. No. 5,545,723 and 4,914,033; Examples below); receptor binding (e.g., U.S. Pat. No. 5,545,723 and Examples below; STAT activation (e.g., Examples below); activation of IFN-B2 responsive genes (e.g., an interferon-stimulated response element (ISRE) is operably linked to a reporter gene, such as luciferase, as shown in the Examples below); phosphorylation of a type I receptor (Examples below); antiproliferative (U.S. Pat. No. 4,914,033, assessing ability of IFN-β2 to inhibit replication of cell lines; Examples below); immunomodulatory (U.S. Pat. No. 4,914,033, antibody-dependent cellular cytotoxicity; Noronha et al., J. Neuroimmunol., 46:145-154, 1993, inhibition of T-lymphocytes and their production of IFN-gamma (“IFN-γ”); experimental allergic encephalomyelitis (“EAE”) (e.g., Louboutin et al., Acta Neurol. Scand., 88:97-99, 1993; Rott et al., Eur. J. Immunol., 23:1745-1751, 1993; Examples below).

A mammalian IFN-β2 of the present invention, fragments, or substituted polypeptides thereof, can also comprise various modifications, where such modifications include lipid modification, methylation, phosphorylation, glycosylation, covalent modifications (e.g., of an R group of an amino acid), amino acid substitution, amino acid deletion, or amino acid addition. Modifications to the polypeptide can be accomplished according to various methods, including recombinant, synthetic, chemical, etc.

Polypeptides of the present invention (e.g., full-length, fragments thereof, mutations thereof) can be used in various ways, e.g., in assays, as immunogens for antibodies as described below, as biologically-active agents (e.g., having one or more of the activities associated with an IFN-β2 of the present invention).

A polypeptide coding for an IFN-β2 of the present invention, a derivative thereof, or a fragment thereof, can be combined with one or more structural domains, functional domains, detectable domains, antigenic domains, and/or a desired polypeptide of interest, in an arrangement which does not occur in nature, i.e., not naturally occurring. A polypeptide comprising such features is a chimeric or fusion polypeptide. Such a chimeric polypeptide can be prepared according to various methods, including, chemical, synthetic, quasi-synthetic, and/or recombinant methods. A chimeric nucleic acid coding for a chimeric polypeptide can contain the various domains or desired polypeptides in a continuous (e.g., with multiple N-terminal domains to stabilize or enhance activity) or interrupted open reading frame, e.g., containing introns, splice sites, enhancers, etc. The chimeric nucleic acid can be produced according to various methods. See, e.g., U.S. Pat. No. 5,439,819. A domain or desired polypeptide can possess any desired property, including, a biological function such as signaling, growth promoting, cellular targeting (e.g., signal sequence, targeting sequence, such as targeting to the endoplasmic reticulum or nucleus), etc., a structural function such as hydrophobic, hydrophilic, membrane-spanning, etc., receptor-ligand functions, and/or detectable functions, e.g., combined with enzyme, fluorescent polypeptide, green fluorescent protein, (Chalfie et al., Science, 263:802, 1994; Cheng et al., Nature Biotechnology, 14:606, 1996; Levy et al., Nature Biotechnology, 14:610, 1996), etc. A domain can also be an immunoglobulin, e.g., to enhance stability, etc., such as an immunoglobulin heavy, light chain, and/or Fc region, or an epitope tag sequence.

In addition, a polypeptide, or a part of it, can be used as a selectable marker when introduced into a host cell. For example, a nucleic acid coding for an amino acid sequence according to the present invention can be fused in-frame to a desired coding sequence and act as a tag for purification, selection, or marking purposes. The region of fusion can encode a cleavage site to facilitate expression, isolation, purification, etc.

An IFN-β2 polypeptide of the present invention, or a fragment thereof, can also be combined with other cytokines, such as interferons, to make chimeric or hybrid IFN-β2s. Such hybrid IFN-β2s can be between any class of interferon, including alpha, omega, gamma, epsilon, trophoblast, fetal, etc. Hybrids (and muteins as discussed above) can be made which have reduced or restricted activities as compared to the hybrids from which they are produced, e.g., restricted to cell growth regulatory activity, antiviral activity, or immunomodulatory activity. See, e.g., U.S. Pat. No. 4,758,428 for hybrids between fibroblast interferon and alpha-interferon, e.g., using about amino acids 47-187, 74-187, 1-73, etc., of fibroblast interferon.

A polypeptide according to the present invention can be produced in an expression system, e.g., in vivo, in vitro, cell-free, recombinant, cell fusion, etc., according to the present invention. Modifications to the polypeptide imparted by such systems include glycosylation, amino acid substitution (e.g., by differing codon usage), polypeptide processing such as digestion, cleavage, endopeptidase or exopeptidase activity, attachment of chemical moieties, including lipids and phosphates, etc.

A polypeptide according to the present invention can be recovered from natural sources, transformed host cells (culture medium or cells) according to the usual methods, e.g., methods applied to other interferons and other recombinant proteins, including, detergent extraction (e.g., non-ionic detergent, Triton X-100, CHAPS, octylglucoside, Igepal CA-630), phase extraction, n-butanol extraction, ammonium sulfate or ethanol precipitation, acid extraction, anion or cation exchange chromatography, phosphocellulose chromatography, Sephadex, hydrophobic interaction chromatography, hydroxyapatite chromatography, lectin chromatography, gel electrophoresis, affinity chromatography, SDS PAGE, controlled pore glass chromatography (CPG), antibody affinity chromatography, gel filtration, blue sepharose, phenyl-agarose chromatography, CPG and zinc-chelate chromatography (e.g., Heine and Billiau, Methods in Enzymology, 78 (Part A): 448-456, 1981), Cibacron Blue F3GA-Agarose and HPLC (Kenny et al., Methods in Enzymology, 78 (Part A):435-447, 1981). See, also, Innis and McCormick, Methods in Enzymology, 119:397-403, 1986; Dembinski and Sulkowski, Preparative Biochemistry, 16:175-186, 1986; Friesen et al, Methods in Enzymology, 78 (Part A):4330-435, 1981); Pestka et al., Annu. Rev. Biochem., 56:727-77, 1987. Protein refolding steps can be used, as necessary, in completing the configuration of the mature protein.

The present invention also relates to nucleic acids, such as DNAs and RNAs coding for IFN-β2 polypeptides, and fragments thereof, of the present invention. An IFN-β2 nucleic acid, or fragment thereof, is a nucleic acid having a nucleotide sequence obtainable from a natural source. It therefore includes naturally occurring, normal, naturally occurring mutant, and naturally occurring polymorphic alleles (e.g., SNPs), etc. Natural sources include, e.g., living cells obtained from tissues and whole organisms, tumors, cultured cell lines, including primary and immortalized cell lines.

A nucleic acid sequence of the invention can contain the complete coding sequence as shown in FIG. 1, degenerate sequences thereof, and fragments thereof. A nucleic acid according to the present invention can also comprise a nucleotide sequence which is 100% complementary, e.g., an anti-sense, to any nucleotide sequence mentioned above and below.

A nucleic acid, i.e., a polymer of nucleotides or a polynucleotide, according to the present invention can be obtained from a variety of different sources. It can be obtained from DNA or RNA, such as polyadenylated mRNA, e.g., isolated from tissues, cells, or whole organism. The nucleic acid can be obtained directly from DNA or RNA, or from a cDNA library. The nucleic acid can be obtained from a cell or tissue (e.g., from an embryonic or adult cardiac cells or tissues) at a particular stage of development, having a desired genotype, phenotype etc.

As described for the IFN-β2 polypeptide described above, a nucleic acid comprising a nucleotide sequence coding for a polypeptide according to the present invention can include only coding sequence; a coding sequence and additional coding sequence (e.g., sequences coding for leader, secretory, targeting, enzymatic, fluorescent or other diagnostic peptides), coding sequences and non-coding sequences, e.g., untranslated sequences at either a 5′ or 3′ end, or dispersed in the coding sequence, e.g., introns. A nucleic acid comprising a nucleotide sequence coding without interruption for a polypeptide means that the nucleotide sequence contains an amino acid coding sequence for an IFN-β2, with no non-coding nucleotides interrupting or intervening in the coding sequence, e.g., absent intron(s). Such a nucleotide sequence can also be described as contiguous. A genomic DNA coding for a human, mouse, or other mammalian interferons, etc., can be obtained routinely.

A nucleic acid according to the present invention also can comprise an expression control sequence operably linked to a nucleic acid as described above. The phrase “expression control sequence” means a nucleic acid sequence which regulates expression of a polypeptide coded for by a nucleic acid to which it is operably linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can be heterologous or endogenous to the normal gene.

A nucleic acid in accordance with the present invention can be selected on the basis of nucleic acid hybridization. The ability of two single-stranded nucleic acid preparations to hybridize together is a measure of their nucleotide sequence complementarity, e.g., base-pairing between nucleotides, such as A-T, G-C, etc. The invention thus also relates to nucleic acids, and their complements, which hybridize to a nucleic acid comprising a nucleotide sequence as set forth in FIG. 1. A nucleotide sequence hybridizing to the latter sequence will have a complementary nucleic acid strand, or act as a template for one in the presence of a polymerase (i.e., an appropriate nucleic acid synthesizing enzyme). The present invention includes both strands of nucleic acid, e.g., a sense strand and an anti-sense strand.

Hybridization conditions can be chosen to select nucleic acids which have a desired amount of nucleotide complementarity with the nucleotide sequence set forth in FIG. 1. A nucleic acid capable of hybridizing to such sequence, preferably, possesses, e.g., about 85%, more preferably, 90%, 92%, and even more preferably, 95%, 97%, or 100% complementarity, between the sequences. The present invention particularly relates to nucleic acid sequences which hybridize to the nucleotide sequence set forth in FIG. 1 under low or high stringency conditions.

Nucleic acids which hybridize to IFN-β2 sequences can be selected in various ways. For instance, blots (i.e., matrices containing nucleic acid), chip arrays, and other matrices comprising nucleic acids of interest, can be incubated in a prehybridization solution (6× SSC, 0.5% SDS, 100 μg/ml denatured salmon sperm DNA, 5× Denhardt's solution, and 50% formamide), at 30° C., overnight, and then hybridized with a detectable oligonucleotides probe, (see below) in a hybridization solution (e.g., 6× SSC, 0.5% SDS, 100 μg/ml denatured salmon sperm DNA and 50% formamide), at 42° C., overnight in accordance with known procedures. Blots can be washed at high stringency conditions that allow, e.g., for less than 5% bp mismatch (e.g., wash twice in 0.1× SSC and 0.1% SDS for 30 min at 65° C.), i.e., selecting sequences having 95% or greater sequence identity. Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C.

Whereas high stringency washes can allow for less than 5% mismatch, relaxed or low stringency wash conditions (e.g., wash twice in 0.2× SSC and 0.5% SDS for 30 min at 37° C.) can permit up to 20% mismatch. Another non-limiting example of low stringency conditions includes a final wash at 42° C. in a buffer containing 30 mM NaCl and 0.5% SDS. Washing and hybridization can also be performed as described in Sambrook et al., Molecular Cloning, 1989, Chapter 9.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log₁₀[Na⁺]+0.41(%GC)−600/N where [Na⁺] is the molar concentration of sodium ions, %GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 95%, 97%, 98%, 99%, nucleotide complementarity between the probe (e.g., an oligonucleotide of an IFN-β2) and target nucleic acid.

According to the present invention, a nucleic acid or polypeptide can comprise one or more differences in the nucleotide or amino acid sequence set forth in FIGS. 1 and 2. Changes or modifications to the nucleotide and/or amino acid sequence can be accomplished by any method available, including directed or random mutagenesis.

A nucleic acid coding for a mammalian IFN-β2, such as human IFN-β2 according to the invention can comprise nucleotides which occur in a naturally occurring gene, e.g., naturally occurring polymorphisms, normal or mutant alleles (nucleotide or amino acid), mutations which are discovered in a natural population of mammals, such as humans, monkeys, pigs, mice, rats, or rabbits. By the term “naturally occurring”, it is meant that the nucleic acid is obtainable from a natural source, e.g., animal tissue and cells, body fluids, tissue culture cells, forensic samples. Naturally occurring mutations can include deletions (e.g., a truncated amino- or carboxy-terminus), substitutions, inversions, or additions of nucleotide sequence. These genes can be detected and isolated by nucleic acid hybridization according to methods which one skilled in the art would know. A nucleotide sequence coding for a mammalian IFN-β2 of the invention can contain codons found in a naturally occurring gene, transcript, or cDNA, for example, e.g., as set forth in FIG. 1, or it can contain degenerate codons coding for the same amino acid sequences. For instance, it may be desirable to change the codons in the sequence to optimize the sequence for expression in a desired host.

A nucleic acid according to the present invention can comprise, e.g., DNA, RNA, synthetic nucleic acid, peptide nucleic acid, modified nucleotides, or mixtures. A DNA can be double- or single-stranded. Nucleotides comprising a nucleic acid can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAase H, improved in vivo stability, etc. See, e.g., U.S. Pat. No. 5,378,825.

Various modifications can be made to the nucleic acids, such as attaching detectable markers (avidin, biotin, radioactive elements), moieties which improve hybridization, detection, or stability. The nucleic acids can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Another aspect of the present invention relates to oligonucleotides or nucleic acid probes. Such oligonucleotides or nucleic acid probes can be used, e.g., to detect, quantitate, or isolate a mammalian IFN-β2 nucleic acid in a test sample, or to identify IFN-β2 homologs. In a preferred embodiment, the nucleic acids can be utilized as oligonucleotide probes, e.g., in PCR, differential display, gene chips (e.g., Affymetrix GeneChips; U.S. Pat. No. 5,143,854, U.S. Pat. No, 5,424,186; U.S. Pat. No. 5,874,219; PCT WO 92/10092; PCT WO 90/15070), and other available methods. Detection can be desirable for a variety of different purposes, including research, diagnostic, and forensic. For diagnostic purposes, it may be desirable to identify the presence or quantity of a nucleic acid sequence in a sample, where the sample is obtained from tissue, cells, body fluids, etc. In a preferred method, the present invention relates to a method of detecting a nucleic acid comprising, contacting a target nucleic acid in a test sample with an oligonucleotide under conditions effective to achieve hybridization between the target and oligonucleotide; and detecting hybridization. An oligonucleotide in accordance with the invention can also be used in synthetic nucleic acid amplification such as PCR (e.g., Saiki et al., Science, 241:53, 1988; U.S. Pat. No. 4,683,202; PCR Protocols: A Guide to Methods and Applications, Innis et al., eds., Academic Press, New York, 1990); differential display (See, e.g., Liang et al., Nucl. Acids Res., 21:3269-3275, 1993; U.S. Pat. No. 5,599,672; WO97/18454); linear PCR; or other amplification methods.

Detection can be accomplished in combination with oligonucleotides for other genes, e.g., genes involved in signal transduction, growth, cancer, apoptosis, or any of the genes mentioned above or below, etc. Oligonucleotides can also be used to test for mutations, e.g., using mismatch DNA repair technology as described in U.S. Pat. No. 5,683,877; U.S. Pat. No. 5,656,430; Wu et al., Proc. Natl. Acad. Sci., 89:8779-8783, 1992.

Oligonucleotides of the present invention can comprise any continuous nucleotide sequence of FIG. 1 or a complement thereto, or any of the sequences, or complements thereto as mentioned above. These oligonucleotides (nucleic acid) according to the present invention can be of any desired size, e.g., about 10-200 nucleotides, 12-100, 12-50, 12-25, 14-16, at least about 15, at least about 20, at least about 25, at least about 30, etc. The oligonucleotides can have non-naturally occurring nucleotides, e.g., inosine, AZT, 3TC, etc. The oligonucleotides can have 100% identity or complementarity to a sequence of FIG. 1, or it can have mismatches or nucleotide substitutions, e.g., 1, 2, 3, 4, or 5 substitutions. In accordance with the present invention, the oligonucleotide can comprise a kit, where the kit includes a desired buffer (e.g., phosphate, Tris, etc.), detection compositions, etc. The oligonucleotide can be labeled or unlabeled, with radioactive or non-radioactive labels as known in the art.

Another aspect of the present invention is a nucleotide sequence which is unique to a mammalian IFN-β2. By a unique sequence to an IFN-β2, it is meant a defined order of nucleotides which occurs in IFN-β2, e.g., in the nucleotide sequences of FIG. 1, but rarely or infrequently in other nucleic acids, especially not in an animal nucleic acid, preferably mammal, such as human, rat, mouse, etc. Unique nucleotide sequences include the sequences, or complements thereto, coding for amino acids KHFFGTV, IIFQQRQV, KSLSP, FRANI, AEKLSGT, CLFFVFS, and QGRPLNDMKQELTTEFRSPR, and fragments thereof as shown in FIG. 1. Such sequences can be used as probes in any of the methods described herein or incorporated by reference. Both sense and antisense nucleotide sequences are included. A unique nucleic acid according to the present invention can be determined routinely. A nucleic acid comprising such a unique sequence can be used as a hybridization probe to identify the presence of, e.g., human or mouse IFN-β2, in a sample comprising a mixture of nucleic acids, e.g., on a Northern blot. Hybridization can be performed under high stringent conditions (see, above) to select nucleic acids (and their complements which can contain the coding sequence) having at least 95% identity (i.e., complementarity) to the probe, but less stringent conditions can also be used. A unique IFN-β2 nucleotide sequence can also be fused in-frame, at either its 5′ or 3′ end, to various nucleotide sequences as mentioned throughout the patent, including coding sequences for other parts of IFN-β2, enzymes, GFP, etc., expression control sequences, etc.

As already discussed, hybridization can be performed under different conditions, depending on the desired selectivity, e.g., as described in Sambrook et al., Molecular Cloning, 1989. For example, to specifically detect IFN-β2 of the present invention, an oligonucleotide can be hybridized to a target nucleic acid under conditions in which the oligonucleotide only hybridizes to it, e.g., where the oligonucleotide is 100% complementary to the target. Different conditions can be used if it is desired to select target nucleic acids which have less than 100% nucleotide complementarity, at least about, e.g., 99%, 97%, 95%, 90%, 86.4%, 85%, 70%, 67%.

Antisense nucleic acid can also be prepared from a nucleic acid according to the present invention, preferably an anti-sense to a sequence of FIG. 1. Antisense nucleic acid can be used in various ways, such as to regulate or modulate expression of IFN-β2, e.g., inhibit it, to detect its expression, or for in situ hybridization. These oligonucleotides can be used analogously to U.S. Pat. No. 5,576,208. For the purposes of regulating or modulating expression of IFN-β2, an anti-sense oligonucleotide can be operably linked to an expression control sequence.

For the inhibition of IFN-β2, an oligonucleotide can be designed to the corresponding sense position along cDNA. See, e.g., J. Milligan et al., Current Concepts in Antisense Drug Design., J. Med. Chem. 36(14): 1923-1937,1993; Helene and Toulme, Biochim. Biophys. Acta, 1049: 99-125, 1990; Cohen, J. S., Ed., Oligodeoxynucleotides as Antisense Inhibitors of Gene Expression, CRC Press: Boca Raton, Fla., 1987; Crooke, S., Basic Principles of Antisense Therapeutics, Springer-Verlag Berlin, Heidelberg, New York, July 1998. Such oligonucleotides can be nuclease-resistant, e.g., using the various chemical linkages disclosed in U.S. Pat. No. 6,040,296 or in the aforementioned references. A total length of about 35 bp can be used in cell culture with cationic liposomes to facilitate cellular uptake, but for in vivo use, preferably shorter oligonucleotides are administered, e.g., 25 nucleotides.

The nucleic acid according to the present invention can be labeled according to any desired method. The nucleic acid can be labeled using radioactive tracers such as ³²P, ³⁵S, ¹²⁵I, ³H, or ¹⁴C, to mention some commonly used tracers. The radioactive labeling can be carried out according to any method such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a nucleic acid of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

A nucleic acid according to the present invention, including oligonucleotides, anti-sense nucleic acid, etc., can be used to detect expression of IFN-β2 in whole organs, tissues, cells, etc., by various techniques, including Northern blot, PCR, in situ hybridization, differential display, nucleic acid arrays (e.g., “gene chips”), dot blots, etc. Such nucleic acids can be particularly useful to detect disturbed expression, e.g., cell-specific and/or subcellular alterations, of IFN-β2. The levels of IFN-β2 can be determined alone or in combination with other gene products, especially other gene products involved in cytokine production.

A nucleic acid according to the present invention can be expressed in a variety of different systems, in vitro and in vivo, according to the desired purpose. For example, a nucleic acid can be inserted into an expression vector, introduced into a desired host, and cultured under conditions effective to achieve expression of a polypeptide coded for by the nucleic acid. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding nucleic acid is adjacent to a dhfr gene), cycloheximide, cell densities, culture dishes, etc. A nucleic acid can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a nucleic acid of the present invention has been introduced is a transformed host cell. The nucleic acid can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, 293, PAE, human, human fibroblast, human primary tumor cells, testes, glia, neurons, oligodendrocytes, astrocytes, neuroblastoma, glioma, etc., insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic stem cells (e.g., mammalian, such as mouse or human), neuronal stem cells, fibroblasts, muscle cells, cardiac cells, and T-cells.

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, promoters of other genes in the cell signal transduction pathway, MMTV, SV40, trp, lac, tac, or T7 promoters for bacterial hosts; or alpha factor, alcohol oxidase, or PGH promoters for yeast. RNA promoters can be used to produce RNA transcripts, such as T7 or SP6. See, e.g., Melton et al., Nucleic Acids Res., 12(18):7035-7056, 1984; Dunn and Studier, J. Mol. Biol., 166:477-435, 1984; U.S. Pat. No. 5,891,636; Studier et al., Gene Expression Technology, Methods in Enzymology, 85:60-89, 1987.

A nucleic acid or polypeptide of the present invention can be used as a size marker in nucleic acid or protein electrophoresis, chromatography, etc. Defined restriction fragments can be determined by scanning the sequence for restriction sites, calculating the size, and performing the corresponding restriction digest.

An IFN-β2 polypeptide and nucleic acid of the present invention can be “isolated”. By the term “isolated”, it is meant that it is in a form in which it is not found in its original environment or in nature, e.g., more concentrated, more purified, separated from components, present in a lysate of a cell in which a heterologous IFN-β2 gene is expressed. When IFN-β2 is expressed as a heterologous nucleic acid in a transfected cell line, a nucleic acid in accordance with the present invention is introduced into a cell as described above, under conditions in which the nucleic acid is expressed. The term “heterologous” means that the nucleic acid has been introduced into the cell line by the “hand-of-man”. Introduction of a nucleic acid into a cell line is discussed above. The transfected (or transformed) cell expressing the IFN-β2 nucleic acid can be lysed as described in the examples and used in the method as a lysate (i.e., “isolated”) or the cell line can be used intact.

Generally, the term “effective conditions” means, e.g., a milieu in which the desired effect is achieved. Such a milieu, includes, e.g., buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.).

The present invention also relates to a method of modulating, preferably inhibiting, expression of a nucleic acid coding for an IFN-β2 of the present invention, comprising: contacting a cell expressing an IFN-β2 of the present invention with an amount of agent, such as an antisense oligonucleotide or antisense RNA of a IFN-β2, which is effective to sequence-specifically inhibit expression of said nucleic acid.

Sequence-specific inhibition of a nucleic acid can be accomplished conventionally using antisense nucleic acid, such as antisense oligonucleotides or RNA. For example, antisense oligonucleotides, such as phosphodiester or phosphorothioate deoxyoligonucleotides can be designed to specific regions of a IFN-β2 RNA, such as to the translation initiation site, and can then be administered to cells expressing such genes in quantities effective to inhibit their expression. Generally, an antisense nucleic acid is a nucleic acid which is complementary to the sense or coding strand of a given nucleic acid, and as a result are also complementary and thus able to specifically hybridize with mRNA transcripts of the nucleic acid. Preferred antisense oligonucleotides comprise the 5′ region of a target gene, especially the region containing the initiation codon.

To enhance stability, the administered nucleic acid can be modified, e.g., to make it resistant to cellular enzymes, oxidation, reduction, nucleases, etc., or to enhance its uptake into cells. Any suitable modification can be used, including, e.g., phosphorothioates, methylphosphonates, phosphodiester oligonucleotide linked to an acridine intercalating agent and/or a hydrophobic tail, psoralen derivatives, 2′-ribose modifications, pentose sugar derivatives, nitrogen base derivatives, etc. See, e.g., U.S. Pat. No. 5,576,208, 5,744,362, 6,040,296, and 6,046,319 for antisense oligonucleotides, modifications, etc. which can be useful in the invention. In general, an antisense nucleic acid of the present invention can comprise monomers of naturally occurring nucleotides, non-naturally occurring nucleotides, and combinations thereof to enhance cellular uptake and/or stability.

Antisense can be administered as naked nucleic acid, complexed or encapsulated with and by other agents which facilitate its uptake into a cell, injected into cells, or any suitable delivery means.

The present invention also relates to methods of using an IFN-β2 of the present invention, such as human IFN-β2, for treating any conditions, disorders, diseases, etc. for which an IFN-β2 bioactivity is desired. Such methods involve administering an effective amount of an IFN-β2 of the present invention to a host in need of treatment for one or more the following purposes: anti-oncogene regulation, antitumor activity, antiviral activity, cell growth inhibition or antigrowth activity, anti-proliferation (e.g., amounts of IFN-β2 which are effective to inhibit the proliferation of astrocytes), enhancement of cytotoxicity of lymphocytes, immunoregulatory activity, inducement or inhibition of differentiation of target cells, macrophage activation, down-regulation of oncogenes, etc.; immunological effects, such as, reducing antibody formation, increasing cell membrane components (major histocompatability complex, Fc receptor, β2-microglobulin), modulating cell-mediated immunity, increasing cytokine (e.g., interleukin) production, increasing cytotoxic T cell effects, increasing macrophage effects, and increasing natural killing. IFN-β2 can be administered to treat, e.g., cancer, autoimmune disorders, and viral infections. See, e.g., Cirelli and Tyring, Clin Immunbother. 3:27-87, 1995, for various disorders which can be treated with IFN-β2 of the present invention; in particular, see uses for alpha- and beta-interferons.

IFN-β2 can be administered as a polypeptide, or it can be administered as a nucleic acid, e.g., as in gene therapy. When administered as a nucleic acid, it can be provided in any form which is effective to achieve expression, e.g., as naked DNA, as a vector (such as viral vector, e.g., adenovirus), complexed in liposomes or other carrier agents, microbubbles, etc. See, above for more information on administration of nucleic acids, their expression in a host, etc.

Any cancer can be treated in accordance with the present invention, e.g., cervical intraepithelial neoplasia and cervical cancer (e.g., see, DePalo et al., Int. J. Tissue React., 6:523-527, 1984, for dosages, administration routes, regimes, etc.), melanoma and metastatic melanoma (e.g., see, Beiteke et al., Hautarzt, 44:365-371, 1994, for dosages, administration routes, regimes, etc.), hairy cell leukemia, Kaposi's sarcoma, basal cell carcinoma, squamous cell carcinoma, renal cell carcinoma, carcinoid tumors, cutaneous T cell lymphoma, nonHodgkin's lymphoma (for other cancers, see, also, Dorr, Drugs, 45(2):177-211, 1993).

Autoimmune diseases can also be treated in accordance with the present invention, e.g., multiple sclerosis (see, e.g., Yong et al., Neurology, 51:682-689, 1998, for dosages, administration routes, regimes, etc.), rheumatoid arthritis, etc. Multiple sclerosis (“MS”) is an autoimmune disease pathologically characterized by perivascular and periventricular inflammation leading to demyelination, axonal destruction and subsequent gliosis. The hallmark of chronic MS lesions is demyelinated gliotic plaques formed as a result of local astrocytic hypertrophy and hyperplasia. These astrocytic plaques interfere with normal axonal conduction and present a physical barrier to remyelination. Therefore, the factors that inhibit astrocytosis have beneficial therapeutic implications, especially in the latter stages of a disease. Thus, the ability of IFN-β2 to inhibit astrocytes is especially useful to treat MS. Viral disease and infections can also be treated in accordance with the present invention, e.g., human papilloma virus (e.g., Puligheddu et al., Eur. J. Gynaecol. Oncol., 9:161-162, 1988; Costa et al., Cervix, 6:203-212, 1988, for dosages, administration routes, regimes, etc.), condylomata acuminata (Schonfeld et al., Lancet, 1: 1038-1042, 1984, for dosages, administration routes, regimes, etc.), hepatitis B, C, and D, HIV, etc.

By the term “administering”, it is meant that IFN-β2, or other active agent, is delivered to the target, e.g., the tumor, the immune system, the brain lesion (e.g., a site of brain inflammation, such as sites observed in multiple sclerosis or other brain inflammatory conditions), etc. IFN-β2 can be administered to any target (e.g., in vivo, in vitro, or in situ), including cells in culture and hosts having an injury, condition, or disease to be treated, by an effective route suitable to achieve an effect as described above, e.g., an IFN-β2 formulation can be administered by injection directly into, or close by, a target site. It can also be administered topically, enterally, parenterally, intravenously, intramuscularly, subcutaneously, orally, nasally, intracerebrally, intraventricularly, etc., e.g., depending upon the location of the target site to be treated. An IFN-β2 can also be administered as a nucleic acid for uptake by cells. Methods to administer nucleic acid include those described above, and other conventional state-of-the-art techniques.

An effective amount of an IFN-β2 is administered to the target. Effective amounts are such amounts which are useful to achieve the desired effect, preferably a beneficial or therapeutic effect, e.g., an amount effective to inhibit astrocyte proliferation. Such amount can be determined routinely, e.g., by performing a dose-response experiment in which varying doses are administered to target cells to determine an effective amount in achieving the desired purpose, e.g., producing antiviral effect, producing an immunomodulatory effect. Amounts can be selected based on various factors, including the milieu to which the IFN-β2 is administered (e.g., a patient with a multiple sclerosis, animal model, tissue culture cells, etc.), the site of the cells to be treated, the age, health, gender, and weight of a patient or animal to be treated, etc. Useful amounts include, e.g., 1.6 MIU (million International Units according to the international reference standard) and 8 MIU administered subcutaneously on alternate days. By the term “treating”, it is meant any effect that results in the improvement of the condition, disease, disorder, etc.

An effective amount of IFN-β2 can be administered with other effective agents, e.g., effective agents for treating cancer, viruses, MS, hepatitis, and any other conditions which can be treated with IFN-β2. Such agents can be cytotoxic, an antiviral agent, a chemotherapeutic agent, etc.

The present invention also relates to antibodies which specifically recognize an IFN-β2 of the present invention. An antibody specific for IFN-β2 means that the antibody recognizes a defined sequence of amino acids within or including an IFN-β2, e.g., the sequence of FIG. 2. Thus, a specific antibody will generally bind with higher affinity to an amino acid sequence, i.e., an epitope, found in FIG. 2 than to a different epitope(s), e.g., as detected and/or measured by an immunoblot assay or other conventional immunoassay. Thus, an antibody which is specific for an epitope of human IFN-β2 is useful to detect the presence of the epitope in a sample, e.g., a sample of tissue containing human IFN-β2 gene product, distinguishing it from samples in which the epitope is absent. A useful antibody is to the unique C-terminus of IFN-β2, e.g., QGRPLNDMKQELTTEFRSPR, or a fragment thereof. Such antibodies are useful as described in Santa Cruz Biotechnology, Inc., Research Product Catalog, and can be formulated accordingly.

Antibodies, e.g., polyclonal, monoclonal, recombinant, chimeric, humanized, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. For example, for the production of monoclonal antibodies, a polypeptide according to FIG. 2 can be administered to mice, goats, or rabbits subcutaneously and/or intraperitoneally, with or without adjuvant, in an amount effective to elicit an immune response. The antibodies can also be single chain or Fab fragments. The antibodies can be IgM, IgG, subtypes, IgG2a, IgG1, etc. Antibodies, and immune responses, can also be generated by administering naked DNA. See, e.g., U.S. Pat. Nos. 5,703,055; 5,589,466; 5,580,859.

Interferon, or fragments thereof, for use in the induction of antibodies do not need to have bioactivity; however, they must have immunogenic activity, either alone or in combination with a carrier. Peptides for use in the induction of IFN-β2-specific antibodies may have an amino sequence consisting of at least five amino acids, preferably at least 10 amino acids. Short stretches of amino acids, e.g., five amino acids, can be fused with those of another protein such as keyhole limpet hemocyanin, or another useful carrier, and the chimeric molecule used for antibody production. Regions of IFN-β2 useful in making antibodies can be selected empirically, or, e.g., an amino acid sequence of IFN-β2, as deduced from the cDNA, can be analyzed to determine regions of high immunogenicity. Analysis to select appropriate epitopes is described, e.g., by Ausubel, F. M. et al. (1989, Current Protocols in Molecular Biology, Vol. 2. John Wiley & Sons).

Particular IFN-β2 antibodies are useful for the diagnosis of prepathologic conditions, and chronic or acute diseases which are characterized by differences in the amount or distribution of IFN-β2. Diagnostic tests for IFN-β2 include methods utilizing the antibody and a label to detect IFN-β2 in human (or mouse, etc., if using mouse, etc.) body fluids, tissues or extracts of such tissues. Antibodies can be neutralizing and utilized in assays for IFN-β2 activity, e.g., as controls to neutralize the interferon activity.

The polypeptides and antibodies of the present invention may be used with or without modification. Frequently, the polypeptides and antibodies will be labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149; and 4,366,241.

Antibodies and other ligands which bind IFN-β2 can be used in various ways, including as therapeutic, diagnostic, and commercial research tools, e.g., to quantitate the levels of an interferon polypeptide in animals, tissues, cells, etc., to identify the cellular localization and/or distribution of it, to purify it, or a polypeptide comprising a part of it, to modulate the function of it, in Western blots, ELISA, immunoprecipitation, RIA, etc. The present invention relates to such assays, compositions and kits for performing them, etc. Utilizing these and other methods, an antibody according to the present invention can be used to detect IFN-β2 polypeptide or fragments thereof in various samples, including tissue, cells, body fluid, blood, urine, cerebrospinal fluid.

In addition, ligands which bind to an IFN-β2 polypeptide according to the present invention, or a derivative thereof, can also be prepared, e.g., using synthetic peptide libraries or aptamers (e.g., Pitrung et al., U.S. Pat. No. 5,143,854; Geysen et al., J. Immunol. Methods, 102:259-274, 1987; Scott et al., Science, 249:386, 1990; Blackwell et al., Science, 250:1104, 1990; Tuerk et al., 1990, Science, 249:505).

The present invention also relates to an IFN-β2 polypeptide, prepared according to a desired method, e.g., as disclosed in U.S. Pat. No. 5,434,050. A labeled polypeptide can be used, e.g., in binding assays, such as to identify substances that bind or attach to IFN-β2, to track the movement of IFN-β2 in a cell, in an in vitro, in vivo, or in situ system, etc.

A nucleic acid, polypeptide, antibody, IFN-β2, etc., can be isolated. By “isolated” means that the material is in a form in which it is not found in its original environment or in nature, e.g., more concentrated, more purified, separated from component, etc. An isolated nucleic acid includes, e.g., a nucleic acid having the sequence of IFN-β2 separated from the chromosomal DNA found in a living animal, e.g., as the complete gene, a transcript, or a cDNA. This nucleic acid can be part of a vector or inserted into a chromosome (by specific gene targeting or by random integration at a position other than its normal position) and still be isolated in that it is not in a form which it is found in its natural environment. A nucleic acid or polypeptide of the present invention can also be substantially purified. By substantially purified, it is meant that nucleic acid or polypeptide is separated and is essentially free from other nucleic acids or polypeptides, i.e., the nucleic acid or polypeptide is the primary and active constituent.

The present invention also relates to a transgenic animal, e.g., a non-human-mammal, such as a mouse, comprising an IFN-β2. Transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology. See, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Annu. Rev. Genet., 20:465-499, 1986; Askew et al., Mol. Cell. Biol., 13:4115-4124, 1993; Games et al., Nature, 373:523-527, 1995; Valancius and Smithies, Mol. Cell. Biol., 11:1402-1408, 1991; Stacey et al., Mol. Cell. Biol., 14:1009-1016, 1994; Hasty et al., Nature, 350:243-246, 1995; Rubinstein et al., Nucl. Acids Res., 21:2613-2617, 1993. A nucleic acid according to the present invention can be introduced into any non-human mammal, including a mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, Trends in Biotech., 5:13-19, 1987; Clark et al., Trends in Biotech., 5:20-24, 1987); and DePamphilis et al., BioTechniques, 6:662-680, 1988). In addition, e.g., custom transgenic rat and mouse production is commercially available. These transgenic animals can useful animals models to test for IFN-β2 function, as food for a snake, as a genetic marker to detect strain origin (i.e., where an IFN-β2, or fragment thereof has, been inserted), etc. Such transgenic animals can further comprise other transgenes. Transgenic animals can be prepared and used according to any suitable method.

The present invention also relates to a mammalian cell in which expression of a gene coding for an IFN-β2 has been “knocked out” or disrupted. Such gene disruption can be accomplished by an effective means, including, e.g., antisense, or by the insertion of a nucleotide sequence into it which is effective to suppress the gene's expression. The term “gene” is used in this sense to mean the IFN-β2 coding sequence as it exists on the chromosome, and is to include its promoter sequence and other regulatory regions.

The invention especially relates to a mammal containing one or more cells in which expression of the gene is functionally inactivated or disrupted. Functional inactivation or disruption refers, e.g., to a partial or complete reduction of the expression of at least a portion of a polypeptide encoded by an endogenous IFN-β2 gene of a single cell, selected cells, or all of the cells of a mammal. The term “knockout” is a synonym for functional inactivation of the gene.

In one embodiment, a gene targeting strategy is utilized that facilitates the introduction of a desired nucleotide sequence into a IFN-β2 gene. The gene targeting strategy preferably utilizes double reciprocal recombination and a positive selectable marker to assist in the insertion of the nucleotide sequence into a target nucleic acid. The target nucleic acid is preferably a gene, more preferably a gene at its particular chromosomal locus. The desired nucleotide sequence is inserted into the gene in such a way that the gene is functionally disrupted, i.e., its expression is partially or completely reduced.

In one aspect of the invention, a targeting vector is employed to insert a selectable marker into a predefined position of an IFN-β2 gene. The position is selected to achieve functional disruption of the gene upon insertion of the selectable marker. For such purposes, a preferred embodiment is a recombinant nucleic acid molecule comprising: (1) a 5′ nucleotide sequence which is effective to achieve homologous recombination at a first predefined position of a mammalian IFN-β2 gene, operably linked to (2) the 5′ terminus of a first selectable nucleotide sequence which confers a first selection characteristic on a cell in which it is present, and (3) a 3′ nucleotide sequence which is effective to achieve homologous recombination at a second predefined position of the mammalian IFN-β2 gene, e.g., IFN-β2 gene, operably linked to the 3′ terminus of the first selectable nucleotide sequence. The recombinant nucleic acid molecule is effective to achieve homologous recombination in a mammalian chromosome at predefined location. Fragments of the targeting vector are also within the scope of the invention, e.g., recombinant nucleic acid molecules comprising elements (1) and (2), or comprising elements (2) and (3), etc.

The term recombinant refers, e.g., to a nucleic acid molecule which has been modified by the hand-of-man, e.g., comprising fragments of nucleic acid from different sources or a nucleic acid molecule from one source which has been engineered. Thus, the nucleic acid molecule is recombinant, e.g., because it comprises nucleotide sequences from a mammalian IFN-β2 gene and selectable marker gene. A molecule is also recombinant when it contains sequences from the same gene but arranged in a manner that does not occur in nature, i.e., a non-naturally occurring arrangement.

Homologous recombination refers to the process in which nucleic acid molecules with similar genetic information line up side-by-side and exchange nucleotide strands. A nucleotide sequence of the recombinant nucleic acid which is effective to achieve homologous recombination at a predefined position of a target nucleic acid therefore indicates a nucleotide sequence which facilitates the exchange of nucleotide strands between the recombinant nucleic acid molecule at a defined position of a target gene, e.g., a mouse IFN-β2 gene. The effective nucleotide sequence generally comprises a nucleotide sequence which is complementary to a desired target nucleic acid molecule (e.g., the gene locus to be modified), promoting nucleotide base pairing. Any nucleotide sequence can be employed as long as it facilitates homologous recombination at a specific and selected position along the target nucleic acid molecule. Generally, there is an exponential dependence of targeting efficiency on the extent or length of homology between the targeting vector and the target locus. Selection and use of sequences effective for homologous recombination is described, e.g., in Deng and Capecchi, Mol. Cell. Biol., 12:3365-3371, 1992; Bollag et al., Annu. Rev. Genet., 23:199-225, 1989; Waldman and Liskay, Mol Cell. Biol., 8:5350-5357, 1988.

An aspect of the present invention is to suppress or functionally disrupt expression of a IFN-β2 gene. The phrases “disruption of the gene”, “gene disruption,” “suppressing expression,” “gene suppression,” “functional inactivation of the gene,” or “functional gene inactivation” refer to modification of the gene in manner which decreases or prevents expression of that gene and/or its product in a cell. The expression of the gene's product can be completely or only partially suppressed, e.g., reduced by 70%, 80%, 85%, 90%, 95%, 99%, or more. A functionally disrupted gene, e.g., a functionally disrupted IFN-β2 gene, includes a modified gene which expresses a truncated polypeptide having less than the entire coding sequence of the wild-type gene. Such a gene is illustrated FIG. 1. A gene can also be functionally disrupted by affecting its mRNA structure in such a way to create an untranslatable message, e.g., frame-shift, decreased stability, etc.

In accordance with the present invention, a IFN-β2 gene is modified in such a manner which is effective to disrupt expression of the corresponding gene product. Thus, e.g., a functionally disrupted recombinant IFN-β2 gene does not express a functional IFN-β2 polypeptide or expresses a functional IFN-β2 polypeptide at levels which are less than wild-type levels of IFN-β2, e.g., reduced by 70%, 80%, 85%, 90%, 95%, 99%, or more. By “not functional” or “functionally inactive” IFN-β2 polypeptide, it is meant, e.g., that the IFN-β2 lacks one or more its bioactivities. The gene can be modified in any effective position, e.g., enhancers, promoters, regulatory regions, noncoding sequences, coding sequences, introns, exons, etc., so as to decrease or prevent expression of that gene in the cell. Insertion into a region of a IFN-β2 gene, e.g., a murine IFN-β2 gene, can be accomplished by homologous recombination. A recombinant nucleic acid molecule comprising regions of gene homology and a nucleotide sequence coding for a selectable marker gene is inserted into the promoter and/or coding region and/or noncoding regions of a IFN-β2, whereby expression of the gene is functionally disrupted. When this knockout construct is then inserted into a cell, the construct can integrate into the genomic DNA. Thus, progeny of the cell will only express only one functional copy of the gene; the other copy will no longer express the gene product, or will express it at a decreased level, as the endogenous nucleotide sequence of the gene is now disrupted by the inserted nucleotide sequence. If desired, the functional gene can be inactivated in a second analogous step.

The nucleotide sequence effective for homologous recombination can be operably linked to a nucleotide sequence, preferably a selectable marker nucleotide sequence or gene, which is to be inserted into the desired target nucleic acid.

The recombinant nucleic acid is preferably inserted into a cell with chromosomal DNA that contains the endogenous gene to be knocked out. In the cell, the recombinant nucleic acid molecule can integrate by homologous recombination with the DNA of the cell in such a position so as to prevent or interrupt transcription of the gene to be knocked out. Such insertion usually occurs by homologous recombination (i.e., regions of the targeting vector that are homologous or complimentary to endogenous DNA sequences hybridize to each other when the targeting vector is inserted into the cell; these regions can then recombine so that part of the targeting vector is incorporated into the corresponding position of the endogenous DNA).

As discussed, one or more nucleotide sequences can be inserted into a gene to suppress its expression. It is desirable to determine the presence of the inserted nucleotide sequence in the gene. This can be accomplished in various ways, including by nucleic acid hybridization, antibody binding to an epitope encoded by the inserted nucleic acid, or by selection for a phenotype of the inserted sequence. Accordingly, such an inserted nucleotide sequence can be referred to as a first selectable nucleotide sequence. A first selectable nucleotide sequence preferably confers a first selection characteristic on a cell in which it is present. By the phrase “selection characteristic,” it is meant, e.g., a characteristic which is expressed in a cell and which can be chosen in preference to another or other characteristics. The selectable nucleotide sequence, also known as selectable marker gene, can be any nucleic acid molecule that is detectable and/or assayable after it has been incorporated into the genomic DNA of the mammal. The selection characteristic can be a positive characteristic, i.e., a characteristic which is expressed or acquired by cells and whose presence enables selection of such cells. A positive selection characteristic can enable survival of the cell or organism, e.g., antibiotic resistance, ouabain-resistance (a gene for a ouabain-resistant sodium/potassium ATPase protein). Examples of positive selection characteristics and a corresponding selection agent include, e.g., Neo and G418 or kanomycin; Hyg and hygromycin, hisD and histidinol; Gpt and xanthine; Ble and bleomycin; Hprt and hypoxanthine. See, e.g., U.S. Pat. No. 5,464,764 and Capecchi, Science, 244:1288-1292, 1989. The presence of the selectable gene in the targeted sequence can also be identified by using binding ligands which recognize a product of the selectable gene, e.g., an antibody can be used to identify a polypeptide product coded for by the selectable gene, an appropriate ligand can be used to identify expression of a receptor polypeptide coded for by the selectable gene, or by assaying for expression of an enzyme coded for by the selectable gene. Preferably, the selectable marker gene encodes a polypeptide that does not naturally occur in the mammal.

The selectable marker gene can be operably linked to its own promoter or to another promoter from any source that will be active or can easily be activated in the cell into which it is inserted. However, the selectable marker gene need not have its own promoter attached, as it may be transcribed using the promoter of the gene into which it is inserted. The selectable marker gene can comprise one or more sequences to drive and/or assist in its expression, including, e.g., ribosome-recognition sequences, enhancer sequences, sequences that confer stability to the polypeptide or RNA, and/or a polyA sequence attached to its 3′ end to terminate transcription of the gene.

A positive selectable marker facilitates selection for recombinants in which the positive selectable marker has integrated into the target nucleic acid by homologous recombination. A gene targeting vector in accordance with the present invention can also further comprise a second selection characteristic coded for by a second selectable gene to further assist in the selection of correctly targeted recombinants. A negative selection marker permits selection against cells in which only non-homologous recombination has occurred. In one preferred embodiment, the second selectable marker gene confers a negative selection characteristic upon a cell in which it has been introduced. Such negative selection characteristics can be arranged in the targeting vector in such a way that it can be utilized to discriminate between random integration events and homologous recombination. By the term negative selection, it is meant a selection characteristic which, when acquired by the cell, results in its loss of viability (i.e., it is lethal to the cell). A nucleoside analog, gancyclovir, which is preferentially toxic to cells expressing HSV tk (herpes simplex virus thymidine kinase), can be used as a negative selection agent, as it selects for cells which do not have an integrated HSV tk selectable marker. FIAU (1,2-deoxy-2-fluoro-.alpha.-d-arabinofuransyl-5-iodouracil) can also be used as a selection agents to select for cells lacking HSV tk. Other negative selectable markers can be used analogously. Examples of negative selection characteristics and a corresponding thymidine kinase (HSV tk) and acyclovir, gancyclovir, or FIAU; Hprt and 6-thioguanine or 6-thioxanthine; diphtheria toxin; ricin toxin; cytosine deaminase and 5-fluorocytosine.

The negative selectable marker is typically arranged on the gene targeting vector 5′ or 3′ to the recombinogenic homology regions so that double-crossover replacement recombination of the homology regions transfers the positive selectable marker to a predefined location on the target nucleic acid but does not transfer the negative selectable marker. For example, a tk cassette can be located at the 3′ end of a murine gene, about 150 base pairs from the 3′ stop codon. More than one negative selectable marker can also be utilized in a targeting vector. The positioning of, for example, two negative selection vectors at the 5′ and 3′ ends of a targeting vector further enhances selection against target cells which have randomly integrated the vector. Random integration sometimes results in the rearrangement of the vector, resulting in excision of all or part of the negative selectable marker prior to random integration. When this occurs, negative selection cannot be used to eliminate those cells which have incorporated the targeting vector but by random integration rather than homologous recombination. The use of more than one negative selectable marker substantially enhances the likelihood that random integration will result in the insertion of at least one of the negative selectable markers. For such purposes, the negative selectable markers can be the same or different.

The use of a positive-negative selection scheme reduces the background of cells having incorrectly integrated targeted construct sequences. Positive-negative selection typically involves the use of two active selectable markers: (1) a positive selectable marker (e.g., neo) that can be stably expressed following random integration or homologous targeting, and (2) a negative selectable marker (e.g., tk) that can only be stably expressed following random integration. By combining both positive and negative selection, host cells having the correctly targeted homologous recombination event can be efficiently obtained. Positive-negative selection schemes are described, e.g., in U.S. Pat. No. 5,464,764; WO 94/06908. It is recognized, however, that one or more negative selectable markers are not required to carry out the present invention, e.g., produce a transgenic animal in which an IFN-β2 gene is functionally inactivated or disrupted.

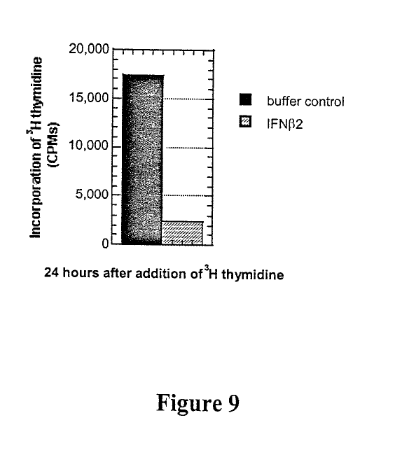

A recombinant nucleic acid molecule according to the present invention can also comprise all or part of a vector. A vector is, e.g., a nucleic acid molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, ΦX174, pBK Phagemid, pNH8A, pNH16a, pNH18Z, pNH46A (Stratagene); Bluescript KS⁺II (Stratagene); ptrc99a, pKK223-3, pKK233-3, pDR540, pRIT5 (Pharmacia). Eukaryotic: PWLNEO, pSV2CAT, pOG44, pXT1, pSG (Stratagene), pSVK3, PBPV, PMSG, pSVL (Pharmacia). However, any other vector, e.g., plasmids, viruses, or parts thereof, may be used as long as they are replicable and viable in the desired host. The vector can also comprise sequences which enable it to replicate in the host whose genome is to be modified. The use of such vector can expand the interaction period during which recombination can occur, increasing the targeting efficiency. An example of a gene targeting vector that can be used in accordance with the present invention is described in Molecular Biology, ed. by Ausubel, F.M., et al., Unit 9.16, FIG. 9.16.1 (pNTK).

In accordance with an aspect of the present invention, the function of an IFN-β2 gene can be disrupted or knocked out by the insertion of an exogenous or heterologous sequence into it, interrupting its function. For example, the exogenous or heterologous sequence can be inserted into a region of the IFN-β2 gene before its first start codon. The nucleotide sequence coding for a selectable characteristic can be inserted into the IFN-β2 gene in such a manner by homologous recombination so that it is operably linked to the endogenous promoter of the IFN-β2 gene. Upon integration of the selectable marker gene into the desired predefined position of the IFN-β2 gene, expression of the selectable characteristic is driven by the endogenous IFN-β2 gene promoter, permitting its detection into those cells into which it has integrated.

The selectable marker gene can also be integrated at positions downstream of 3′ to the first start codon of the IFN-β2 gene. The IFN-β2 gene can be integrated out-of-reading frame or in-reading frame with the IFN-β2 polypeptide so that a fusion polypeptide is made, where the fusion polypeptide is less active than the normal product. By detecting only those cells which express the characteristic, cells can be selected which contain the integrated sequence at the desired location. A convenient way of carrying out such selection is using antibiotic resistance. In the examples below, neomycin resistance is utilized as the selectable characteristic. Cells grown in the presence of a toxic concentration of neomycin will normally die. Acquisition of the neomycin resistance gene by homologous recombination rescues cells from the lethal effect, thereby facilitating their selection.

The IFN-β2 gene is knocked out or functionally interrupted by the integration event. The insertion of the selectable gene ahead of the IFN-β2 coding sequence effectively isolates it from a promoter sequence, disabling its expression. If the selectable gene contains a transcription terminator, then transcription of the gene using the IFN-β2 promoter will terminate immediately after it and will rarely result in the transcription of the IFN-β2 coding sequence. The IFN-β2 gene can also be knocked out by a deletion without a replacement, such as a site-directed deletion of a part of the gene. Deleted regions can be coding regions of regulating regions of the gene.

A IFN-β2 gene can be modified at any desired position. It can be modified so that a truncated IFN-β2 polypeptide is produced having one or more activities of the complete IFN-β2 polypeptide.

If desired, the insertion(s) can be removed from the recombinant gene. In the example, a neomycin cassette replaced exons of the mouse IFN-β2 gene to functionally inactivate it. The neomycin cassette can be subsequently removed from the IFN-β2 gene, e.g., using a recombinase system. The Cre-lox site-specific recombination system is especially useful for removing sequences from a recombinant gene. To utilize the Cre-lox system, recombinase recognition sites are integrated into the chromosome along with the selectable gene to facilitate its removal at a subsequent time. For guidance on recombinase excision systems, see, e.g., U.S. Pat. Nos. 5,626,159, 5,527,695, and 5,434,066. See also, Orban, P. C., et al., “Tissue-and Site-Specific DNA Recombination in Transgenic Mice”, Proc. Natl. Acad. Sci. USA, 89:6861-6865, 1992; O'Gorman, S., et al., “Recombinase-Mediated Gene Activation and Site-Specific Integration in Mammalian Cells”, Science, 251:1351-1355, 1991; Sauer, B., et al., “Cre-stimulated recombination at loxP-Containing DNA sequences placed into the mammalian genome”, Nucl. Acids Res., 17(1):147-161, 1989.

For other aspects of the nucleic acids, reference is made to standard textbooks of molecular biology. See, e.g., Davis et al., Basic Methods in Molecular Biology, Elsevir Sciences Publishing, Inc., New York, 1986; Hames et al., Nucleic Acid Hybridization, IL Press, 1985; Sambrook et al., Molecular Cloning, CSH Press, 1989; Howe, Gene Cloning and Manipulation, Cambridge University Press, 1995.

### EXAMPLES

Expression and Purification of IFN-β2. Purified IFN-β2 was compared to IFN-β1b by SDS-PAGE. IFN-β2 has an apparent molecular weight of 26 kDa as determined by SDS-PAGE, while IFN-β1b has an apparent molecular weight of approximately 20.5 kDa. Method: PCR primers (5′-GGA ATT CCT ACT ACC TCG GGC TTC TAA-3′ and 5′-GCG CGC GCA TAT GCT AGA TTT GAA ACT GAT TAT-3′) were designed to amplify the coding region of IFN-β2, minus the signal sequence, from a human genomic DNA preparation for subsequent ligation into an IPTG inducible pet5a expression vector (Promega Corp). After induction, IFN-β2 was isolated from E. coli inclusion bodies and solubilized using Zwittergent 3-14 (Russell-Harde et al., J. Interferon Cytokine Res., 15, 31-37, 1995). Purification of IFN-β2 from solubilized inclusion bodies was achieved by using ion exchange chromatography followed by size exclusion chromatography. The 26 kDa band, corresponding to IFN-β2, was eluted from the SDS-PAGE gel and analyzed by N-terminal protein sequencing. The first 10 amino acids corresponded to those expected for IFN-β2. Furthermore, cyanogen bromide digestion was performed yielding several fragments which were sequenced and found to have the predicted protein sequences, demonstrating that the full protein has been successfully expressed and purified.

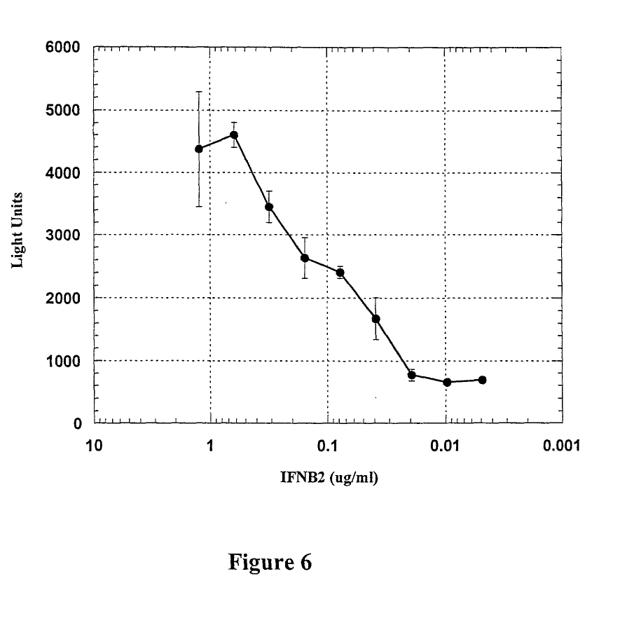

Activation of an Interferon Dependent ISRE-Luciferase Reporter by IFN-β2. T98G cells were transfected with a plasmid containing an ISRE-Luciferase construct and a stable clone expressing the construct was isolated. 3×10⁴ cells were plated overnight and purified IFN-β2 was added at the indicated concentrations. After four hours, the cells were assayed for luciferase activity using a luciferase assay kit as described in the kit protocol (Promega Cat. #E1501). IFN-β2 specifically activated the interferon dependent ISRE reporter. See FIG. 6. Utilizing this assay, IFN-β2 demonstrated functional properties similar to those of IFN-β1b.

Inhibition of Binding of IFN-β2 to the Human Type I Interferon Receptor with an anti-IFN-β2 Mouse Polyclonal Antibody. A peptide corresponding to the unique C-terminal region of IFN-β2 (KLSKQGRPLNDMKQELTTEFR) was synthesized, coupled to KLH and used to immunize Swiss-Webster mice for a total of four immunizations over two months. After immunization, sera was collected and shown to contain antibodies which specifically bind IFN-β2. Furthermore, the anti-IFN-β2 sera blocked the induction of an IFN dependent ISRE-Luciferase reporter by IFN-β2. Utilizing this assay, IFN-β2 demonstrated functional properties similar to those of IFN-β1b.

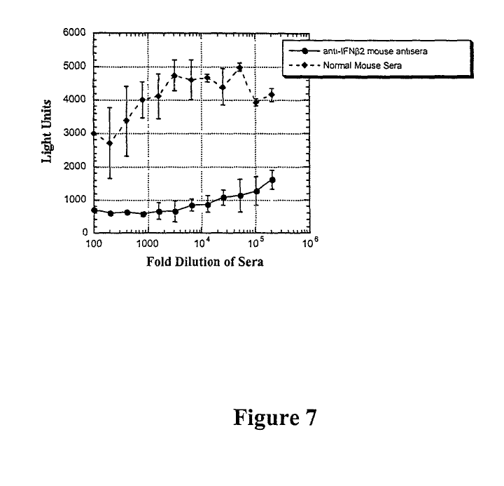

Method: 3×10⁴ cells were plated overnight and 20 ng of IFN-β2 was added either in the presence of anti-IFN-β2 sera or normal mouse sera for four hours and then assayed for the presence of induced luciferase using a luciferase assay kit and the standard protocol from Promega Corp. See FIG. 7.

The Effect of IFN-β1b and IFN-β2 on Human HT1080 Cell Proliferation. IFN-β1b and IFN-β2 have an antiproliferative effect in both the HT1080 cells and HT1080IFNAR2c cells. This was evident in both the Alamar Blue assay panels (FIGS. 8A and B) as well as by visual inspection. The antiproliferative effect correlated with an increase in receptor number as evidenced by an increased effect in the HT1080IFNAR2c cells, which have five times the number of IFN binding sites, when compared to the HT1080 cells. Utilizing this assay, IFN-β2 demonstrated functional properties similar to those of IFN-β1b.

Method: HT1080IFNAR2c cells are HT1080 cells which over express IFNAR2c. These cells exhibit a five fold greater number of binding sites for IFN than the parental HT1080 cells. 2-5×10⁴ cells/ml were plated overnight and either unstimulated, or stimulated with 1 μg/ml, 500 ng/ml, 200 ng/ml or 50 ng/ml of IFN-β2. However, HT1080 cells were stimulated with 1 μg/ml, 500 ng/ml or 200 ng/ml of IFN-β, while HT1080IFNAR2c cells were stimulated with 500 ng/ml, 200 ng/ml or 50 ng/ml of IFN-β. Alamar Blue (U.S. Pat. No. 5,501,959) was used to measure cell proliferation using the standard protocol, and photos were taken at each time point of a representative field. Media containing interferon was replaced daily. All treatments were done in triplicate. See FIG. 8.

Antiproliferative Activities of IFN-β2 on Human HT1080 Cells as Measured by Short Term ³[H] Thymidine Incorporation. Incorporation of ³[H] thymidine was measured 48 hours after addition of IFN-β2 (striped column) or buffer control (filled column). ³[H] thymidine incorporation is presented as CPM incorporated/10⁶cells. Data (FIG. 9) represent mean values of n=3 and variations between replicates were less than 15%. IFN-β2 decreased thymidine incorporation significantly, e.g., by about 86%. Utilizing this assay, IFN-β2 demonstrated functional properties similar to those of IFN-β1b.

Methods: Cells were seeded (2×10⁴ cells/well) in a 24-well cell culture plate, incubated overnight and then stimulated with IFN-β2 (1 μg/ml) for 24 hours. Cells were then incubated in complete media containing ³[H] thymidine ([methy -³H] thymidine, specific activity=40-60 Ci/mmol, Amersham Life Science) and harvested after 24 hours. Cells were washed with phosphate-buffered saline (PBS), followed by 10% trichloroacetic (TCA) acid and 100% ethanol. Prior to determining incorporation of radioactivity, cells were solubilized in IM potassium hydroxide and mixed with Ecolume scintillation fluid.

Type I IFN Receptor Activation by IFN-β2. IFN-β2 induced tyrosine phosphorylation of the IFNAR2c receptor chain of the human type I IFN receptor. Cells were either unstimulated (unstim.) or stimulated with human IFN-α2, IFN-β1b or IFN-β2 (1000-2000 relative units/10⁶ cells for 15 minutes). Phosphorylation was observed in the presence but not absence of interferon. Utilizing this assay, IFN-β2 demonstrated functional properties similar to those of IFN-β1b.

Methods: Daudi cells expressing IFNAR2c (5×10⁷ cells) were solubilized in lysis buffer (20 mM Tris-HCl, pH 7.5, containing 1% Nonidet-40 (v/v) (NP-40), 150 mM sodium chloride, 1mM EDTA, 2.5% glycerol (v/v), 1.0 mM sodium fluoride, 1.0 mM sodium orthovanadate, 1.0 mM phenylmethysulfonyl fluoride (PMSF), 0.5 μg/ml leupeptin and 5.0 μg/ml trypsin inhibitor) for 30 minutes at 4° C. and insoluble material was removed by centrifugation. For immunoprecipitation IFNAR2c antisera (+) or negative control antisera (−) was added to each sample, incubated overnight, mixed with Protein-G agarose (Boehringer-Mannheim), and resolved by SDS-PAGE (10% Novex gels). Proteins were transferred to polyvinylidene difluoride filters (Pro-Blot) and incubated in blocking buffer (20 mM Tris-HCl, pH 7.5 containing 0.1% Tween 20 (v/v), 150 mM sodium chloride, 1 mM EDTA, 1.0 mM sodium fluoride, 1.0 mM sodium orthovanadate, 1.0 mM PMSF, 0.5 μg/ml leupeptin and 5.0 μg/ml trypsin inhibitor) overnight at 4° C., incubated with an anti-phosphotyrosine antibody (ab PY99, Santa Cruz Biotechnology, Inc. Santa Cruz, Calif.) antibody and washed in blocking buffer. Following washing, the membrane was incubated with a specific second antibody (1:1000 dilution) coupled to horseradish peroxidase (HRP) for 1 hour, washed 3 times in blocking buffer and developed using a chemiluminescent detection method (Pierce).

STAT1 and STAT2 Activation in Daudi Cells by Stimulation with IFN-β2. Daudi cells were stimulated with IFN-β1b or IFN-β2 (1000-2000 relative units/10⁶ cells) for 15 minutes, solubilized in lysis buffer and STAT1 and STAT2 were immunoprecipitated. Following immunoprecipitation, tyrosine phosphorylation of STAT1 and STAT2 was detected using a phosphotyrosine specific antibody, for both IFN types. Utilizing this assay, IFN-β2 demonstrated functional properties similar to those of IFN-β1b.

Methods: Daudi cells (1×10⁷ cells) were solubilized in lysis buffer (20 mM Tris-HCl, pH 7.5, containing 1% Nonidet-40 (v/v) (NP-40), 150 mM sodium chloride, 1 mM EDTA, 2.5% glycerol (v/v), 1.0 mM sodium fluoride, 1.0 mM sodium orthovanadate, 1.0 mM phenylmethysulfonyl fluoride (PMSF), 0.5 μg/ml leupeptin and 5.0 μg/ml trypsin inhibitor) for 30 minutes at 4° C. and insoluble material was removed by centrifugation. For immunoprecipitation, STAT1 and 2 antibodies (Stat1 p91 and Stat2 (C-20) respectively, Santa Cruz Biotechnology, Inc. Santa Cruz, Calif.) were added to each sample, incubated overnight, mixed with Protein-G agarose (Boehringer-Mannheim), and resolved by SDS-PAGE (10% Novex gels). Proteins were transferred to polyvinylidene difluoride filters (Pro-Blot) and incubated in blocking buffer (20 mM Tris-HCl, pH 7.5 containing 0.1% Tween 20 (v/v), 150 mM sodium chloride, 1 mM EDTA, 1.0 mM sodium fluoride, 1.0 mM sodium orthovanadate, 1.0 mM PMSF, 0.5 μg/ml leupeptin and 5.0 μg/ml trypsin inhibitor) overnight at 4° C., incubated with an anti-phosphotyrosine antibody (PY99, Santa Cruz Biotechnology, Inc. Santa Cruz, Calif.) and washed in blocking buffer. Following washing, the membrane was incubated with a specific second antibody (1:1000 dilution) coupled to horseradish peroxidase (HRP) for 1 hour, washed 3 times in blocking buffer and developed using a chemiluminescent detection method (Pierce).

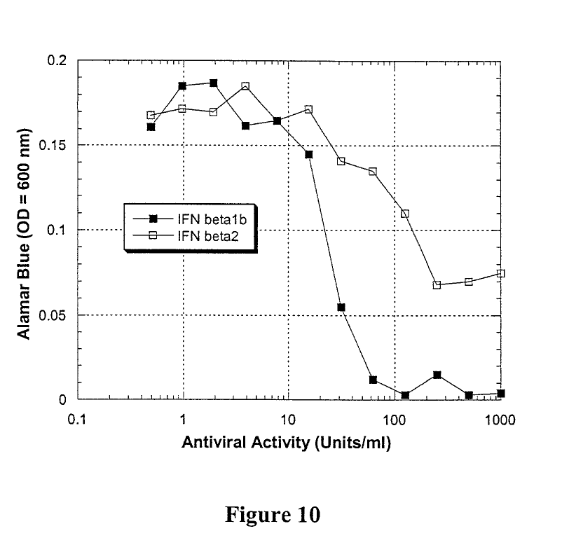

Antiviral Activity of IFN-β2 and IFN-β1b. Human WISH cells were stimulated with either IFN-β1b or IFN-β2 followed by infection with vesicular stomatitis virus (VSV). Viral cytopathic effect (CPE) was measured using the redox dye Alamar Blue. Units of antiviral activity corresponding to IFN-β1b are plotted along the X-axis. Specific antiviral activity of IFN-β2 was determined to be 4.0-8.0×10⁶ International Units (“IU”) per mg. See FIG. 10. Utilizing this assay, IFN-β2 demonstrated functional properties similar to those of IFN-β1b.

Methods: WISH cells (30,000 cells/well) were plated in 96-well Falcon microtiter plates and allowed to attach overnight. Cells were stimulated with IFN-β1b (1000 IU in first well; specific activity=2.5×10⁷ IU/ml) or IFN-β2 (1 μg in first well), diluted 1:1 across the plate, for 6 hours followed by addition of VSV (7×10³ plaque forming units/well, (PFUs)) for 18 hours. Following incubation, media was removed, and 100 μl of Alamar Blue (Biosource International) (1:10 dilution of the manufactures supplied stock solution in media) was added to each well. After incubation for 30-60 minutes at 37° C., CPE was determined by measuring the absorbance at 600 nm.

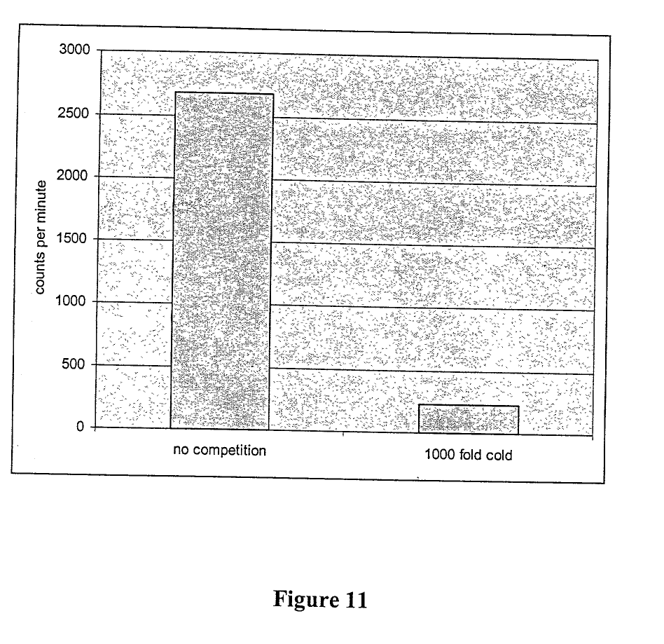

IFN-β2 Competes with IFN-α2 for Binding to the Type I IFN Receptor on HT1080 cells. 1×10⁶ HT1080 cells were incubated for 90 minutes with 15 ng/ml ³²P-labeled IFN-α2 (Pestka Biomedical #51100) in full cell culture media (10% FBS, DMEM). After incubation, the cells were washed twice with cell culture media, solubilized in 1% SDS, mixed with scintillation fluid and counted. 15 μg/ml IFN-β2 competed more than 90% of the labeled IFN-β2 bound to HT1080 cells. Assays were performed in triplicate and standard deviations were less than 10 percent. See FIG. 11.

Competitive Binding of IFN-β2 to the Type I IFN Receptor on Daudi cells. Competitive ligand binding assays are performed with a phosphorylated form of IFN-α2. The ligand is phosphorylated (specific activities of 60-62 μCi/μg) as described in Croze, E., et al., J. Biol. Chem., 271:33165-33168, 1996. Binding data are analyzed described in Scatchard, G., Ann. N.Y. Acad. Sci., 51, 660-672, 1965. Nonspecific binding is determined in the presence of 100-fold excess of unlabeled IFN. Competitive binding of different IFNs is determined by incubating increasing amounts of unlabelled IFN-α2, IFN-β1b or IFN-β2 with a constant amount of phosphorylated IFN-α2. Utilizing this assay, IFN-β2 demonstrates functional properties similar to those of IFN-β1b.

IFN-β Specific Assembly of the Type I IFN Receptor. IFN-β1b interacts with the type I IFN receptor in a manner distinguishable from IFN-α2. Utilizing this assay, IFN-β2 demonstrates functional properties similar to those of IFN-β1b.

Methods: Cells (1×10⁸) are stimulated with IFNs at a concentration of 200 IU/10⁶ cells at 37° C. for 15 minutes in a CO₂ incubator. After treatment, cells are quickly harvested at 4° C. by centrifugation (3000×g, 3 minutes) and immediately solubilized in ice cold lysis buffer (100 mM Tris, pH 8.0, containing 150 mM NaCl, 10% glycerol (v/v), 1% NP-40 (v/v), 1 mM orthovanadate, 1 mM sodium pyrophosphate, 1 mM sodium fluoride, 1 mM EDTA, 1 mM phenylmethylsulfonyl flouride, 5 μg/ml leupeptin and 5 μg/ml trypsin inhibitor). The lysate is centrifuged (16,000×g, 30 minutes) at 4° C. and the supernatant is collected. Cell lysates are immunoprecipitated using anti-IFNAR1 antibodies, as described in Croze, E., et al., J. Biol. Chem., 271, 33165-33168, 1996, or IFNAR2.2 rabbit polyclonal antisera (10 μl of antisera/10⁸ cells), followed by SDS-PAGE analysis using Novex 8% Tris-glycine gels. After electrophoresis, proteins are transferred to polyvinylidene fluoride (PVDF) filters (Pro-Blot) and blocked with 20 mM Tris, pH 8.0, containing 150 mM NaCl, 1 mM orthovanadate, 1 mM sodium pyrophosphate, 1 mM sodium fluoride, 1 mM PMSF, and 0.1 % Tween 20 overnight at ambient temperature. The filters are subsequently incubated with antibodies directed against IFNAR1 (40H2, 0.1 μg/ml, as described in Croze, E., et al., J. Biol. Chem., 271, 33165-33168, 1996) or IFNAR2 (10 μl antisera/10 ml blocking buffer) for 2 to 3 hours at ambient temperature followed by four 10 minute washes with blocking buffer. The washed filter is then incubated with the corresponding horseradish peroxidase (HRP) conjugated second antibody for 2 to 3 hours at ambient temperature, washed and developed using chemiluminescence (Enhanced Chemiluminescence Detection Kit, Pierce).

Preferential Induction of Genes by Different Classes of Interferons. Interferons induce overlapping, distinct sets of genes in cultured cells. Daudi or HT1080 cells are stimulated with either human IFN-α2 (1000 IU/10⁶ cells), IFN-β1b (1000 IU/10⁶ cells), IFN-γ(1000 IU/10⁶ cells), or IFN-β2 (1000 IU/10⁶ cells) for 17 hours, and whole cell pellets collected and processed for TaqMan® analysis as described in the TaqMan® Gold RT-PCR Protocol Manual, Applied Biosystems, Perkin-Elmer Corporation P/N 402876 Rev. A 1997. For RNase protection assays of gene expression, cells are stimulated and harvested as described in Sandhya, R. et al., J. Biol. Chem., 271, 22878-22884, 1996. Genes preferentially induced by IFN-β1b are normalized to the expression of ISG 6-16, a gene induced equally by IFN-α and IFN-β. Utilizing this assay, IFN-β2 demonstrates functional properties similar to those of IFN-β1b.

Antiproliferation of human fetal astrocytes in response to IFN-β2. Astrocytes contribute to the development of MS legions, and here we show that IFN-β2 inhibits the proliferation of human fetal astrocytes in vitro. This observation suggests that IFN-β2 can act as a growth regulator of astrocyte proliferation and therefore prevent the formation of reactive gliotic lesions in MS. Utilizing this assay, IFN-β2 demonstrated functional properties similar to those of IFN-β1b.

### Methods

(A) Preparation of astroglial cultures: Astrocyte enriched cultures from fetal human brains were prepared from 2 different fetal brains of 17-22 weeks gestation. Tissue was obtained from Advanced Bioscience Resource Inc. following legal therapeutic abortion. After the meninges were removed, the brains were dissected and dissociated into single cell suspension by gentle pipeting followed by passing them through sieves. Cells were resuspended in Iscove's media containing 10% FCS in the presence of an antibiotic cocktail containing penicillin, streptomycin and fungizone, and microglia were removed every day for a week by a differential adhesion technique. Astrocytes were then grown for at least 8-10 weeks and fed twice a week. Contaminating microglia, neurons and oligodendrocyte progenitors cannot survive these long-term culture conditions. At the end of this period cultures were stained with GFAP, O4 and nestin antibodies and confirmed to be more than 95% pure astrocytes. Cultures were frozen in liquid N₂ before they were used for proliferation assay.

(B) Proliferation assay: Astrocytes were started from the frozen stock and grown in the above-described media for at least two passages before they were used for the proliferation assay. Cells were plated in 96-well plates at 2×10⁴ cells/ml with or without 10 ng/ml EGF (R&D Systems). The assay was performed in low serum media (2% FCS). Cultures were treated with IFN-β2 (1 mg/ml stock) or a buffer control at the indicated dilutions. After 4 days of incubation cultures were incubated overnight with ³H-Thymidine and the plates were frozen before harvesting.

Activity of IFN-β2 in Rodent Models of Multiple Sclerosis. Experimental allergic encephalomyelitis (EAE) is widely used as an animal model for multiple sclerosis (Swanborg, G., Clin. Immunol. Immunopathol., 77, 4-13, 1995; Martin, R. and McFarland, H., Springer Semin. Immunopathol., 18, 1-24, 1996). IFN-β1b shows in vivo efficacy in these relevant MS models. Utilizing these models, IFN-β2 demonstrates functional properties similar to those of IFN-β1b.

### Methods

(A) Passive Transfer Experimental Allergic Encephalomyelitis in SJL Mice

Animals and materials: 8 week old female SJL mice (Jackson Laboratories); RPMI 1640, with L-glutamine and 25 mM HEPES, 1×, 0.1 micron filtered (Life Technologies, Cat #22400-089); FBS, defined (Hyclone, heat inactivated, Cat #SH30070.01); MEM Nonessential amino acids solution, 10 mM, 100× (Life Technologies, Cat #11140-050); 2-mercaptoethanol, 1000×, 5.5×10⁻² M in D-PBS (Life Technologies, Cat #21985-023); Penicillin/Streptomycin, 10,000 U/μg per ml (Bio-Whittaker, Cat #17-602 E); Hank's Balanced Salt Solution, 1×, 0.1 micron filtered (Life Technologies; Cat #24020-117); Experiment: 8-week-old female SJL mice are immunized with 0.1 ml subcutaneous (divided between base of tail and upper back) injection containing 150 μg proteolipid protein (“PLP”) in complete Freund's adjuvant (“CFA”) with 200 μg M. tuberculosis H37Ra (ground). 11 days later, axial, brachial and inguinal lymph node cells are excised from the mice and cultured at 6×10⁶ cells/ml in the following media (to 450 ml RPMI 1640 (with L-glutamine plus HEPES) add 50 ml FBS, 0.455 ml 2-mercaptoethanol, 5.0 ml Pen/Strep and 5.0 ml non-essential amino acids. Add PLP to the cells to obtain a final concentration of 50 μg/ml. Cells are incubated for 72 hours at 37° C., 7% CO₂. Cells are harvested and washed twice in HBSS. Viability of the lymph node cells is assessed by Trypan Blue exclusion. The concentration of lymph node cells is adjusted to 4×10⁷ cells per ml. 2×10⁷ lymph node cells are injected intraperitoneally (dose volume=0.5 ml) per mouse into naive 8 week old female SJL mice. The mice are weighed and scored daily. Treatment with IFN-β2 and IFN-β1b is administered as needed. Clinical evaluation (EAE score/symptoms): 0/normal; 1/limp tail; 2/difficulty righting; 3/incomplete paralysis of one or both hind limbs; 4/complete paralysis of one or both hind limbs; 5/immobile, moribund or dead.

(B) Acute Experimental Allergic Encephalomyelitis in Lewis Rats Animals and materials: female Lewis rats (Charles River), immunized at 8 weeks of age; spinal cord homogenate preparation (from male Hartley guinea pigs, Simonsen Labs, Gilroy):

500-700 gram guinea pigs are euthanized with CO₂. The spinal cords are removed using sharp bone scissors to cut the vertebrae, rinsed in saline, blotted once, and stored at −80° C. until the day of use. Spinal cords are then weighed and homogenized with saline at 1 g/ml of saline; antigen emulsion: guinea pig spinal cord homogenate is mixed 1:1 with CFA (Difco, Detroit, Mich.) with 1 mg/ml Mycobacterium tuberculosis (ground with a mortar and pestle). 0.05 ml is injected into each hind limb footpad for a total of 0.1 ml per rat. Experiment: rats are immunized with a single bolus injection on day 1. Rats are weighed and scored daily. Treatment with IFN-β2 and IFN-β1b is administered as needed. Clinical Evaluation (EAE score/symptoms): 0/normal; 1/limp tail; 2/incomplete paralysis of one or both hind limbs; 3/complete paralysis of one hind limb or both hind limbs can move but do not help in movement of the body; 4/complete paralysis of both hind limbs; 5/complete paralysis of hind limbs and weakness of one or both forelimbs or moribund, or death.

The preceding description, utilize the present invention to its fullest extent. The preceding preferred specific embodiments are, therefore, to be construed as merely illustrative, and not limiting the remainder of the disclosure in any way whatsoever. The entire disclosure of all applications, patents and publications, cited above and in the figures are hereby incorporated by reference in their entirety.

What is claimed:

1. A pharmaceutical composition useful in treating multiple sclerosis in mammals, which composition comprises a pharmaceutically acceptable excipient and a therapeutically effective amount of a human IFN-β2 polypeptide, a biologically-active fragment thereof, or a biologically-active derivative thereof.

2. The pharmaceutical composition of claim 1 wherein the mammal in need thereof is a human.

3. A method of administering to a mammal in need thereof a pharmaceutical composition useful in multiple sclerosis in mammals, which composition comprises a pharmaceutically acceptable excipient and a therapeutically effective amount of a human IFN-β2 polypeptide, a biologically-active fragment thereof, or a biologically-active derivative thereof.

4. The method of claim 3 wherein the mammal in need thereof is a human.
